(19) United States
(12) Patent Application Publication
(10) Pub. No.: US 2012/0083669 A1
(43) Pub. Date: Apr. 5, 2012

(54) Personal Nutrition and Wellness Advisor

(76) Inventor: Nabil M. Abujbara, Irvine, CA (US)

(21) Appl. No.: 13/252,620

(22) Filed: Oct. 4, 2011

Related U.S. Application Data
(60) Provisional application No. 61/389,479, filed on Oct. 4, 2010.

(51) Int. Cl. A61B 5/00

(52) U.S. Cl. 600/300

(57) ABSTRACT

Personal nutrition and wellness advisor system comprised of at least one computing platform, medical and nutrition knowledge databases, food nutrient databases, receives and analyzes each user's initial personal and health-related information to intelligently estimate his initial energy & nutrient budgets and physical activity needs; interactively constructs and presents to the user personalized energy and nutrient content-based, location-based, activity-based, and event-based ranked available food item lists and recipes to encourage the user to be physically active and to consume food items containing the most needed nutrients; tracks the user food consumption, physical activity, updates to energy and nutrient budget balances, any physiological parameter measurements, any taken medication types, doses, and times, and any other external events which may cause changes to user-specific health-related variables; and automatically varies the types, rankings, and/or portions of the food items in the recommended available food item list based on the results of tracked activities.

### CROSS REFERENCE TO RELATED APPLICATION

This application claims the benefit of U.S. Provisional Patent Application No. 61/389,479, filed on Oct. 4^{th}, 2010, entitled “Personalized Nutrition and Wellness Advice and Self-management method and system”, the disclosure of which is incorporated herein by reference in its entirety.

### BACKGROUND OF THE INVENTION

1. Field of the Disclosure

The present disclosure relates generally to computer-assisted health management systems and specifically to personal nutrition and wellness advisor systems and methods.

2. Description of the Related Art

There is increasing scientific evidence showing a direct relationship between the alarming global increases in the prevalence of human chronic diseases (such as diabetes, hypertension, dyslipidemia, congestive heart failure, obesity, some types of cancer, and others) and malnutrition, insufficient physical activities, and other unhealthy lifestyle habits. Not changing these unhealthy dietary and lifestyle habits can lead to prolonged periods of undetected and unmanaged health problems; resulting in serious long-term health complications, which would cause significant reduction in patient quality of life while increasing the financial burdens on patients and the global healthcare systems.

It is encouraging that significant government and public funded research has shown that the incidence of these diseases can be decreased and the resulting long-term complications may be prevented, or at least delayed, when people play an active role in self-managing their health on a regular basis. However, such active self-management of one's health requires having the knowledge and discipline to perform a large number of interdependent tasks at various times during each day, such as:

1. Maintaining balanced energy and balanced nutrient budgets by:

Knowing the daily-recommended quantities of energy, macronutrients, and micronutrients needed for maintaining one's healthy body weight and for obtaining adequate nutrition. Such quantities must be based on personal attributes such as the person's age, weight, height, gender, ethnicity, activity level, genetics, any present or predisposition to chronic diseases, and any taken medications.

Knowing how to count the energy, macronutrient, and micronutrient contents of all food and beverage items available for consumption at or away from home.

Consuming the appropriate types and portions of food and beverages to assure obtaining the minimum requirements of macronutrients and micronutrients without exceeding the recommended energy budget.

Keeping count of energy and micronutrients spent while performing physical activities, and making adjustments to the remaining food item selections in order to make up for the spent energy and nutrients.

2. Managing medications: If there is a need to take medications for the maintenance of health, taking the right types and doses of medications at the prescribed times, being aware of any adverse interactions between the taken medications and foods or beverages consumed, and making the correct eating adjustments to avoid any complications arising from such interactions.

3. Taking regular health status (physiological parameter) measurements by using commercially available Personal Health Devices (PHDs) and interpreting and taking action on the results of said measurements (examples are blood glucose for diabetics and blood pressure, weight and blood viscosity for CHF patients) and communicating the results with healthcare providers (HCPs), as well as

4. Avoiding unhealthy behaviors such as lack of sleep, negative stress, smoking and/or overconsumption of alcohol.

Most people find it difficult to perform all the above mentioned learning, tracking, measuring, recording, and balancing tasks on a daily basis for the rest of their lives; without receiving continuous assistance from nutrition, fitness, and healthcare providers; which is impractical and not financially affordable for most people.

Another potential public health problem may arise from the overconsumption of Multi-Vitamin and Mineral (MVM) supplements. Although many people are aware of the importance of consuming the required amounts of micronutrients through natural food items, a very large number of consumers find it more convenient to take daily doses of MVM supplements available in abundance in specialty stores, wholesale stores, supermarkets, and pharmacies. Although MVM supplementation may be needed for some people with severe shortages of certain micronutrients or who are not able to eat whole foods, consuming excessive amounts of some vitamins (e.g. Vitamin A) and some minerals (e.g. Iron, Copper, Zinc) are known to have toxic effects. Unfortunately, there is insufficient public health education to make people aware of such potential hazards.

Currently, there are several public and private parties offering various solutions attempting to assist people in reaching their healthy body weights, nutrition, fitness, and wellness goals. The following is a list of some examples of present and proposed private and public health educational initiatives and commercial solutions which share the goal of assisting users in self-managing their health:

United States Department of Agriculture (USDA) nutrition and physical activity educational initiatives MyPyramid and MyPlate available to the public at the following web sites: http://www.mypyramidtracker.gov/ and http://www.choosemyplate.gov/index.html. MyPyramid and MyPlate are modeled after the USDA's most recent “Dietary Guidelines for Americans”. The web sites provide an abundance of educational materials and have an interactive tool to calculate the personalized calorie budget for a user based on age, gender, weight, and activity level and another tool that allows users to build up virtual meal plates from stored lists of food items. The tool displays graphical indicators of the nutritional quality of the selected food items as items are added or removed from the plate. Micronutrient intake is assumed to be satisfactory if the user consumes the recommended servings of fruits and vegetables having certain colors (e.g. green, yellow, orange, etc.), grains and fortified foods.

The weight management solution offered by Weight Watchers® removes the burden from consumers of having to count calorie content of foods by utilizing a “Points System” whereby a budget of a number of “Points” per day is given for each customer to consume based on age, height, present weight, and target weight. One “Point” is typically worth between 50 and 60 Kcalories depending on the fiber and fat contents of the specific food item. The customers look up, estimate, or compute with the assistance of a “points calculator” device, the number of points in each meal or snack they consume and keep a record of the tally of total number of points consumed throughout the day. Although the program makes recommendations for healthy eating in magazines and on-line educational resources, customers can basically eat any foods, regardless of the nutritional content, as long as the total number of points in all consumed food items do not exceed the maximum allowed number of points per day.

Jenny Craig® is another weight management solution that removes the burden of counting calories or points from consumers by selling prepackaged foods with premeasured calorie contents for consumption at breakfast, lunch, dinner, and snack times to help people stay within a set budget of calories. They offer a set of on-line tools to assist users in managing their meals, check progress, and get involved in physical activities.

U.S. Pat. No. 7,882,150 titled “Health Advisor” claims methods for presenting “acceptable” and not presenting “unacceptable” menu items available for consumption by a customer at a restaurant or other food service facilities. The determination of acceptability of certain restaurant menu items is based on the nutritional values of the menu items and the dietary requirements of the user.

US patent application number 2011/0009708 A1 titled “System and apparatus for improved nutrition analysis” describes a solution for use by professional fitness coaches and their athlete clients to prepare meal plans appropriate for scheduled physical activities (i.e. training or competition events) during a specific day. Nutritional values of meals in the meal plans are described in terms of total calories and their event-specific macronutrient distribution (i.e. percentages of calories from carbohydrates, proteins and fats). Recommended consumption of the prepared meals and snacks are timed around the particular physical activities.

As can be seen, available solutions focus on assisting people in one or more, but not all required health management tasks mentioned earlier.

In light of the above, what is needed is a personal “Advisor” system that stays aware of each user's health and wellness needs and is always available to give users personalized and timely advice and encouragement relevant to helping them manage their nutrition, physical and mental activities, medications, and self-measurement tasks.

### SUMMARY OF THE INVENTION

The present invention addresses the above-mentioned self-management problems by providing a computer-assisted personal nutrition and wellness advisor system and method, referred to hereinafter as the “Advisor”.

In one embodiment an Advisor method comprises receiving and analyzing each user's initial personal and health-related information to intelligently estimate his initial energy, macronutrient, & micronutrient budgets and physical activity needs; interactively constructing and presenting to the user personalized energy and nutrient content-based, location-based, activity-based, and event-based ranked available food item lists and recipes to encourage the user to consume food items containing the most needed nutrients; tracking the user food consumption, physical activity, updates to energy and nutrient budget balances, any physiological parameter measurements, any taken medication types, doses, and times, and any other external events which may cause changes to user-specific health-related variables; and automatically varying the types, rankings, and/or portions of the food items in the recommended available food item list based on the results of the tracked activities.

Examples of user-specific health-related variables that may change asynchronously are the user's personalized energy, macronutrient and micronutrient intake budgets and medications changed by the user's healthcare provider; newly diagnosed chronic diseases or predisposition to certain chronic diseases; physical condition including any acute sicknesses; food allergies; current physical location; intended destination; available foods at and around the user's location or intended destination; macronutrient and micronutrient contents of each consumed food item; preferred foods; disliked foods; food monetary budget; newly acquired food items; and learnings from continuous advances and discoveries in the nutrition, pharmacology, genetics, nutrigenomics, wellness, and medical fields.

The method will further assist users who take medications in reducing adverse interactions between certain foods and certain medications by managing the times between consuming the certain foods and taking the medications, and by encouraging the user to increase the intake amounts of any micronutrients which may be depleted by certain medications by adding and increasing the rankings of foods and supplements rich in the depleted micronutrients to the recommended food item list.

The method will further limit the consumption of certain food items that contain high amounts of certain nutrients deemed to have harmful effects on the user's health and well being.

In one embodiment a personal nutrition and wellness advisor system comprised of at least one computing platform, one or more medical and nutrition knowledge databases, one or more food nutrient databases, storage means, input means, communications means, and display means receives and analyzes each user's initial personal and health-related information to intelligently estimate his initial energy, macronutrient, & micronutrient budgets and physical activity needs; interactively constructs and presents to the user personalized energy and nutrient content-based, location-based, activity-based, and event-based ranked available food item lists and recipes to encourage the user to consume food items containing the most needed nutrients; tracks the user food consumption, physical activity, updates to energy and nutrient budget balances, any physiological parameter measurements, any taken medication types, doses, and times, and any other external events which may cause changes to user-specific health-related variables; and automatically varies the types, rankings, and/or portions of the food items in the recommended available food item list based on the results of tracked activities.

In one embodiment, a computer-readable medium has computer executable instructions stored thereon, the instructions being executable by one or more computing devices in order to cause the one or more computing devices to perform operations comprising receiving and analyzing each user's initial personal and health-related information to intelligently estimate his initial energy, macronutrient, & micronutrient budgets and physical activity needs; interactively constructing and presenting to the user personalized energy and nutrient content-based, location-based, activity-based, and event-based ranked available food item lists and recipes to encourage the user to consume food items containing the most needed nutrients; tracking the user food consumption, physical activity, updates to energy and nutrient budget balances, any physiological parameter measurements, any taken medication types, doses, and times, and any other external events which may cause changes to user-specific health-related variables; and automatically varying the types, rankings, and/or portions of the food items in the recommended available food item list based on the results of tracked activities.

This is a brief list of the various benefits of the present invention when compared with other health management solutions available today:

1. Automated storage of all user selections takes the burden off the user from having to remember the nutritional contents of each food, and then finding and selecting foods to consume that will satisfy his continuously changing energy and nutrient balances during the day.

2. Encourage the user to consume the recommended food items in order to maintain balance between the energy intake and expenditure, without exceeding or lowering the recommended micronutrient intake requirements.

3. Demonstrated improvement in patients' long-term health outcomes by consuming healthy balanced meals, physical activities, medication adherence and interaction avoidance, and responding to changes in health status as problems arise.

4. More accurate and timely healthcare provider interventions to fine-tune the patients' nutrition and wellness management plans; in response to remote monitoring of patients' health status.

5. Promoting chronic disease self-management, which minimizes the burden on the national healthcare systems leading to major cost reductions.

7. Promoting chronic disease self-management, which minimizes the burden on the national health systems, thus leading to major healthcare cost reductions.

8. Continuous monitoring of patients' health status by healthcare providers, leading to more accurate and timely fine tuning of the patients' Advisor parameters; such as medication types and doses, physical activity plans, recommended food item lists and energy and nutrient budgets, as well as pushing relevant educational multimedia content to the user's favorite multimedia devices.

### BRIEF DESCRIPTION OF THE DRAWINGS

FIG. 1 shows the high-level system architecture and main players in one embodiment of the Advisor System.

FIG. 2 is a block diagram showing the inputs, outputs, and a list of main functions of the Advisor System's Personalized Nutrition and Wellness Recommendation Engine.

FIG. 3 is a flowchart of the setup and initialization steps of one embodiment of the Advisor System.

FIG. 4 is a flowchart of the customization process steps of the Customized Recommended Daily Energy and Nutrient Intake Budget (CRDENIB).

FIG. 5 is Table 1: Sample Estimated Energy Requirement (EER) Equations.

FIG. 6 is Table 2: Daily Resting Energy Expenditure (REE) equations taking diabetes and race into consideration.

FIG. 7 is Table 3: Examples of Calorie Distribution by Major Macronutrient Types.

FIG. 8 is Table 4: Example of Energy Distribution by Major Macronutrient Type for different Calorie Allowances.

FIG. 9 is Table 5: Recommended Daily Micronutrient Intake by Life Stage Group.

FIG. 10 is Table 6: Recommended Daily Micronutrient Intake by Life Stage Group (Upper Limits).

FIG. 11 is a flowchart describing the steps taken to construct a Recommended Available Food Item List (RAFIL).

FIG. 12 is Table 7: Sample entries of Food Nutrient Content Database (NDB).

FIG. 13 is Table 8: Example of an Individualized Food Item Metadata Table.

FIG. 14 is Table 9: Sample Initial RAFIL @ Home.

FIG. 15 depicts steps taken by the present invention to enable the User and/or HCP to make periodic remote adjustments to the Advisor parameters based on measurement data obtained remotely from the user's PHD's and based on updated medical and nutritional information.

FIGS. 16A and 16B depict a flowchart showing the operational steps of the Advisor during a typical day of use.

FIG. 17 is Table 10: Example of real-time RAFIL adjustments during a 24-Hour period.

FIG. 18 is Table 11: Example of Nutrition Budget Computation during a typical day of using the present invention.

FIG. 19 is Table 12: Sample RAFIL when Protein Upper Limit has been met.

FIG. 20 is Table 13: Sample RAFIL when user is at Restaurant X.

FIG. 21 depicts the major system components of a sample implementation of an embodiment of the present invention.

FIG. 22 shows a graphic representation of a typical user experience while utilizing the benefits of a sample implementation of an embodiment of the present invention.

### DETAILED DESCRIPTION OF CERTAIN EMBODIMENTS

The following description is of certain illustrative embodiments of the present invention, and it is understood that the disclosure is not limited to these embodiments, but includes alternatives, equivalents, and modifications such as are included within the scope of the claims.

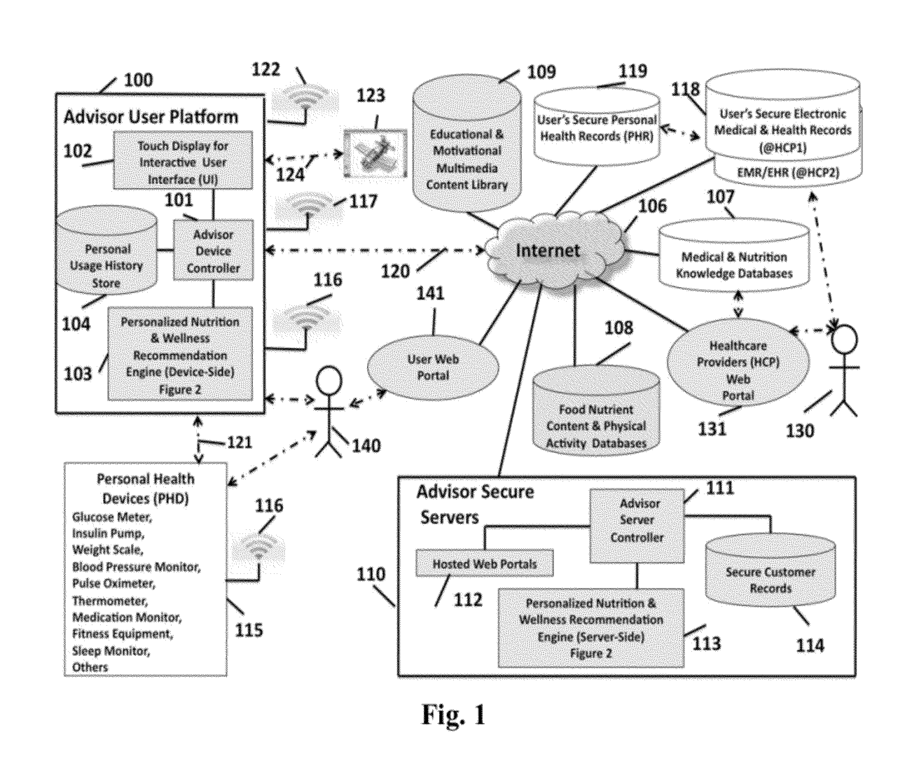

FIG. 1 shows the major components necessary to utilize the benefits of the present invention in one preferred embodiment.

User 140 frequently interacts with Advisor User Platform 100, Personal Health Devices (PHD) 115, User Web Portal 141, and Health Care Provider (HCP) 130 in order to get the personalized advice and assistance he needs to reach and maintain optimum health and wellness goals.

Advisor User Platform 100 presents User 140 with the daily interactive nutrition and wellness ad vice experience, which will be described in the other drawings in detail.

Advisor User Platform 100 can be a mobile phone, Personal Digital Assistant (PDA), computer, tablet, television, automobile display or a dedicated mobile or stationary device capable of providing an interactive user interface.

Advisor User Platform 100 is comprised of several components that are relevant to the present invention.

Advisor Device Controller 101 executes the device-side Personalized Nutrition and Wellness Recommendation Engine (Device-Side) 103 which will be described in more detail with the assistance of the other drawings.

Advisor Device Controller 101 may be an off-the-shelf or custom-built microprocessor or microcomputer system having volatile and non-volatile memories and capable of controlling display and input resources and sufficient processing power to execute the commands required to implement the present invention and storing usage history information in Personal Usage History Store 104 which is comprised of nonvolatile memory devices.

While the present invention can be practiced on devices without touch screen capabilities, Touch Display for Interactive User Interface (UI) 102 is the preferred method for interactions with User 140,

Body Area Network (BAN)/Personal Area Network (PAN)/Local Area Network (LAN) wireless network transceiver 116 capable of communicating with Advisor User Platform 100 over BAN/PAN/LAN network 121,

LAN/Wide Area Network (WAN) wireless network transceiver 117 capable of connecting to resources of the World Wide Web via Internet infrastructure 106 over LAN/WAN network 120, and

Optional feature for mobile versions of Advisor User Platform 100: Global Positioning System (GPS) wireless network transceiver 122, which is capable of connecting to GPS Satellites and cellular towers 123 over GPS network 124 in order to obtain the coordinates of the physical location of User 140.

Personal Health Devices (PHD) 115 are routinely used by User 140 to take measurements of various physiological parameters used as indicators of his health status. Some PHD 115 devices such an insulin pumps, continuous glucose monitors or sleep monitors may be attached to User 140's body, while others are used externally, such as a Blood Glucose Meter, Blood Pressure Monitor or Weight Scale. PHD 115 devices report their measurements visually to User 140 and over BAN/PAN/LAN network 121 to Advisor User Platform 100, which would periodically upload this information over LAN/WAN network 120 to Advisor Secure Servers 110 for storing in Secure Customer Records 114, and to User's Secure Personal Health Records (PHR) 119 to be reviewed by HCP 130 and User 140.

BAN/PAN/LAN network 121 may be wireless (e.g. Bluetooth or Zigbee standards) or wired (e.g. Universal Serial Bus (USB) standard).

User 140 periodically interacts with User Web Portal 141 hosted by Advisor Secure Servers' 110 Hosted Web Portals 112 over Internet 106, which gives him visibility into and control over his health status stored in his Secure Personal Health Records 119, to view personalized multimedia content downloaded from Educational & Motivational Multimedia Content Library 109, and to make modifications to the Advisor User Platform 100 parameters by interacting with Advisor Secure Servers 110. User Web Portal 141 is viewable on any device such as Advisor User Platform 100 or other mobile phone, PDA, or personal computer, connected to Internet 106.

Health Care Provider (HCP) 130 is typically a medical professional such as a physician, nurse, or dietician who utilize the system of the present invention to monitor and manage the health outcome of User 130.

HCP 130 interacts with the system described herein through HCP Web Portal 131, which gives HCP 130 visibility and some control over User 140's health status information stored in an authorized section of User 140's Secure Personal Health Records 119.

Alternatively, HCP 130 can view User 140's information stored in User 140's Electronic Medical/Health Record 118 managed by HCP 130.

HCP 130 also accesses the latest medical and nutrition advances available in Medical & Nutrition Knowledge Bases 107 to help him make any necessary adjustments to User 140's Advisor User Platform 100 settings.

Advisor Secure Servers 110 provide back-end execution and storage services to the personalized nutrition and wellness advice system to complement the functionality of the Advisor User Platform 100.

The Advisor Secure Servers 110 are comprised of one or multiple servers connected to the World Wide Web resources via Internet 106. The servers may be dedicated servers or cloud-based.

The main functional blocks of the Advisor Secure Servers are:

Advisor Server Controller 111 responsible for executing the Personalized Recommendation Engine (Server-Side) functions;

Hosted Web Portals 112 which remotely execute the functions invoked by User Web Portal 141 and HCP Web Portal 131; and

Secure Customer Records 114 where each User's personal information is stored and updated.

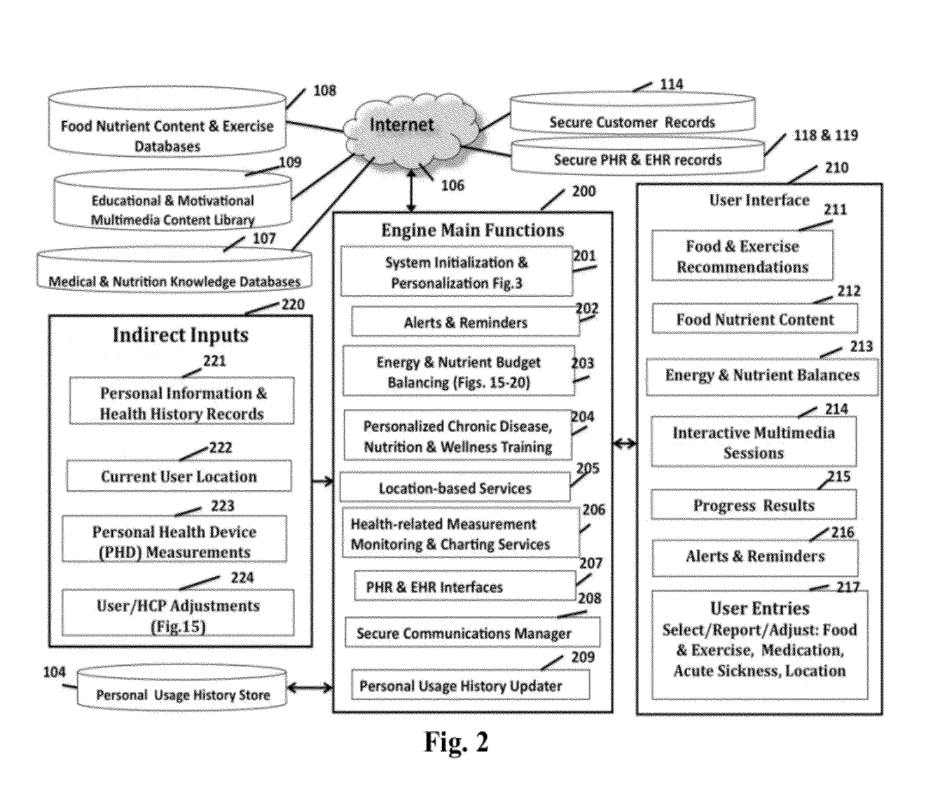

FIG. 2 is a block diagram of the Personalized Nutrition and Wellness Recommendation Engine (Recommendation Engine) 103/113, which contains the core functions of the system described in the present invention.

Recommendation Engine 103/113 is comprised of a set of Engine Main Functions 200, User Interface 210 which supports the main types of interactions between User 140 and the Engine Main Functions 200, and Indirect Inputs 220 to Engine Main Functions 200, which are not directly entered via the user interface 210 but are rather obtained indirectly through events, user actions, user record content changes, and remote interventions as will be described in detail later.

Recommendation Engine 103/113 Main Functions 200 is comprised of these modules:

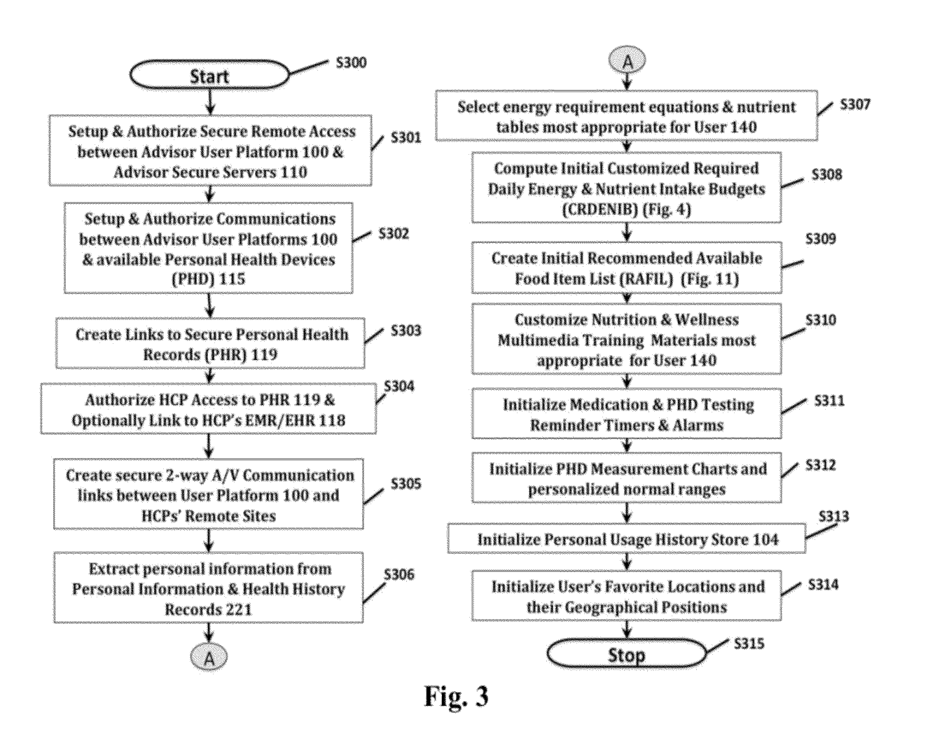

System Initialization & Personalization module 201 is responsible for initializing and personalizing the system parameters, variables, and lists based on personal information of each user. The detailed functionality of this module is described in FIG. 3.

The Alerts & Reminders 202 module is responsible for issuing alerts and reminders on several occasions; such as medication timing reminders, food/medication interactions, exceeding calorie or nutrient upper limits, low consumption of certain micronutrients, out of range health status measurements, adjustments made by HCP 130 in response to health status measurements or new discoveries in the medical or nutrition fields, etc. . . . .

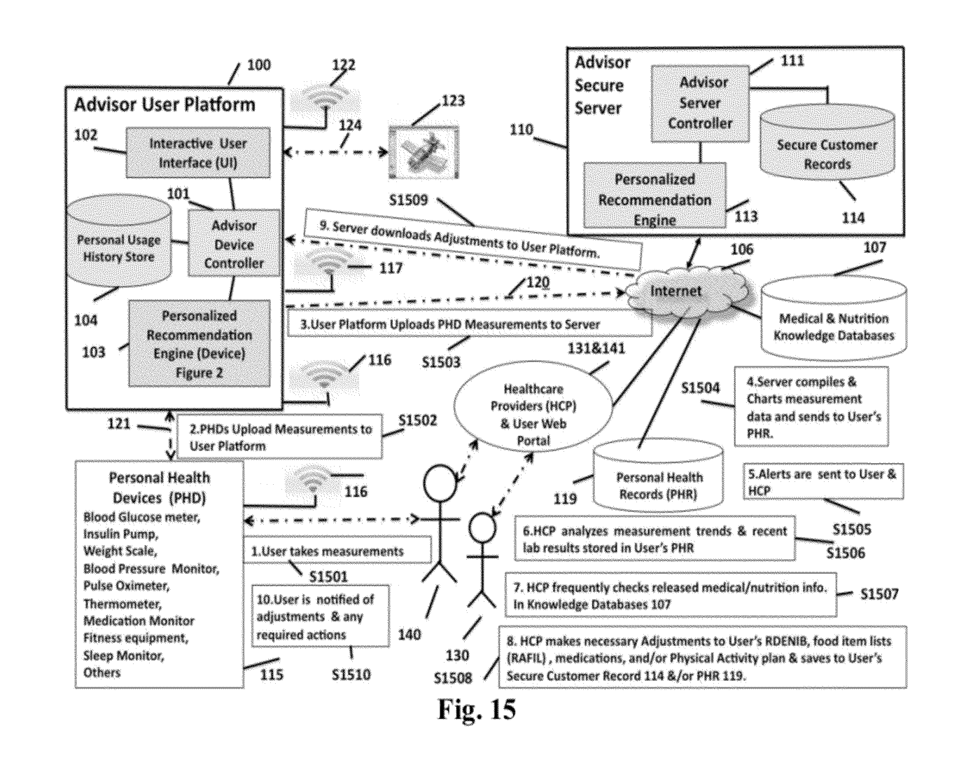

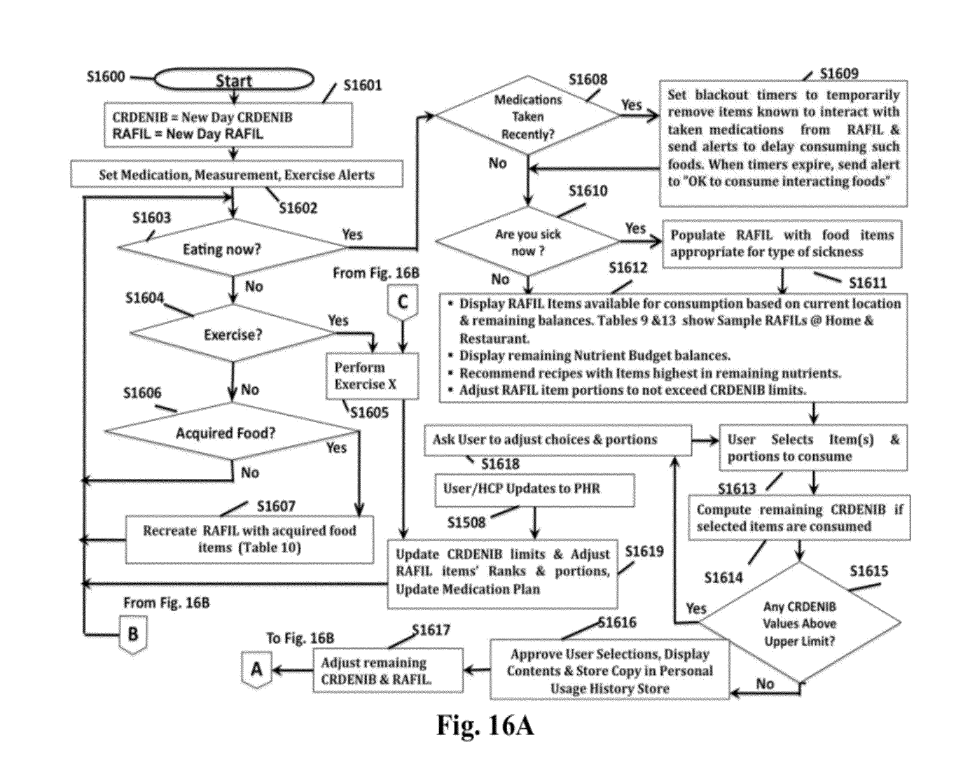

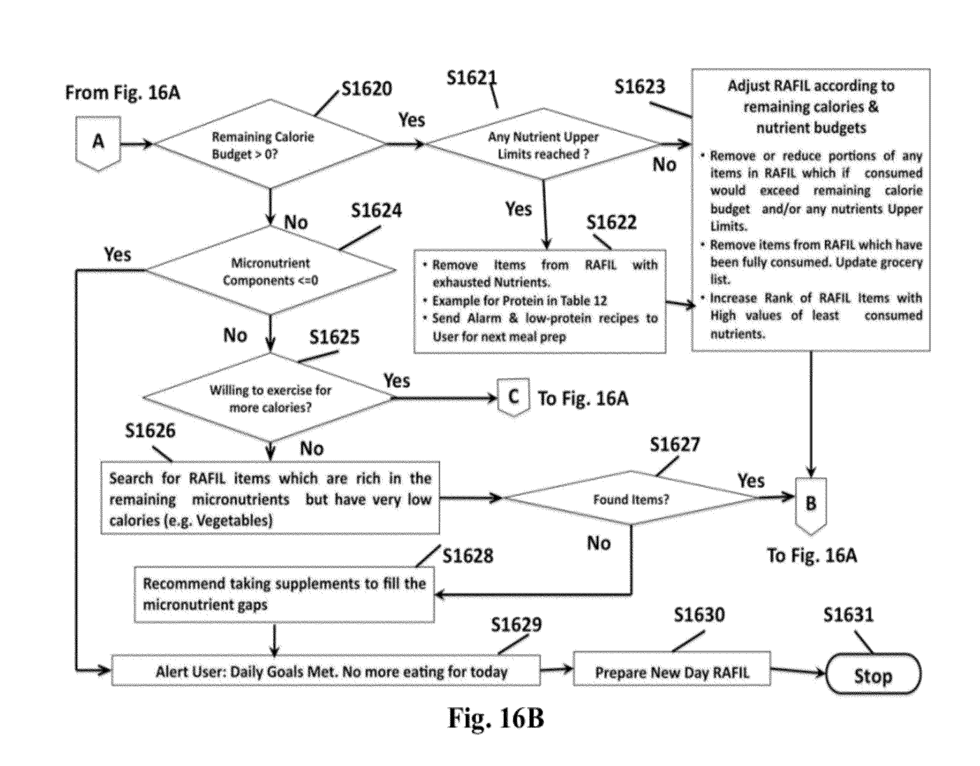

The Energy & Nutrient Budget Balancing module 203 is responsible for keeping track of each user's location, food intake, physical activities, types, doses, and times of taken medications, and PHD measurements. It utilizes this information for dynamically varying the food selections offered to the User 140 with the purpose of balancing the personalized energy and nutrient intake budgets. The varying of food item selections is done in response to several variables, which will be described in more detail in FIGS. 15, 16A, and 16B.

Personalized Chronic Disease, Nutrition & Wellness Training module 204 is responsible for selecting, personalizing, and presenting interactive multimedia content to best satisfy User 140's specific self-management training needs.

Location-based Services module 205 is responsible for determining the location of User 140 (e.g. Home, Office, School, Restaurants, Café's, Fitness Center, etc. . . . ) and making location-based decisions such as available food items list contents and recommended physical activities.

Health-related Monitoring & Charting Services module 206 is responsible for keeping track of User 140's health related physiological measurements and timing, such as blood glucose levels, blood pressure, weight, heart rate, physical activities, food items consumed, and medications taken.

The resulting charts can be utilized by Users and HCP's to spot trends and relationships between certain behaviors, such as eating quality, medication taking, and mental and physical activities on the person's physiological parameter measurements, which are important health status indicators. Knowing how to look for and find the cause and effect of behaviors over health status indicators on the charts, will allow users of the Advisor system to make behavioral adjustments to bring their physiological measurements to the normal ranges shown by the charts. This data is also extremely important when combined with hundreds and thousands of users data and then “mined” by business intelligence tools to predict how effective certain medicines or therapies are, or not.

PHR & EHR Interfaces 207 is responsible for interfacing the Advisor User Platform 100 and Advisor Secure Servers 110 to User 140's Secure Personal Health Records (PHR) 119 and Electronic Medical & Health Records 118 maintained by each of User 140's HCP's 130.

Secure Communications Manager 208 is responsible for establishing secure communications links between the different system components, such as BAN/PAN/LAN network 121 between Advisor User Platform 100 and PHD 115, LAN/WAN 120 network links between Advisor User Platform 100 and Advisor Secure Servers 110, and GPS communication link 124 between Advisor User Platform 100 and GPS Satellite 123.

Personal Usage History Updater 209 is responsible for record keeping all usage history of the Advisor solution and making local copies in Personal Usage History Store 104, Secure Customer Records 114, as well as Secure Personal Health Records 119.

Advisor system component User Interface 210 implements the major graphical or textual views experienced by User 140 when utilizing the features of the present invention in the daily management of his health. It has the following components.

Food and Exercise Recommendations 211 is preferably a graphical visualization of the recommended food items presented to User 140 for food item type(s) and portion selection at meal and snack times. It also provides a personalized set of exercise selections selected to match User 140 capabilities and wellness needs.

Food Nutrient Content 212 displays the total nutrients User 140 is about to consume when a meal or snack are selected from the selections displayed by Food & Exercise Recommendations 211. The displayed nutrient contents of the selected foods are computed from information initially obtained from Food Nutrient Content & Exercise Databases 108 and cached in Personal Usage History Store 225. This aids User 140 in knowing how many calories and nutrients he consumes whenever he eats. Knowing the total amounts of energy and nutrients available in each meal or snack is helpful, for example, when an insulin-dependent User 140 has to compute the insulin dosage needed to counter the hyperglycemic effects of the carbohydrates contained in the food he is about to consume, or has just consumed. It is also helpful for planning the different meals and snacks.

Energy & Nutrient Balances 213 display to the user a running tally of the already consumed and remaining balances of energy and nutrients for the day. This helps User 140 keep track of his food consumption and make better decisions regarding the healthiest food selections by selecting food items having the appropriate amounts of nutrients needed to fulfill his nutritional daily requirements.

User 140 is able to experience personalized Interactive Multimedia Sessions 214 to aid in training him on self-managing his health goals and chronic disease(s). Content shown in Interactive Multimedia Sessions 214 is customized to assist User 140 in optimizing his wellness outcome. This customized content is typically stored in Educational & Motivational Multimedia Content Library 109. Local copies of User 140 favorite or most recently viewed content may be also stored in Personal Usage History Store 104 for faster access.

Progress Monitoring 215 is an on-demand feature whereby User 140 can check his health indicator measurements, foods consumed so far, weight, blood pressure, etc.

Alerts and Reminders 216 are sent during the day when User 140's attention is needed to take actions such as taking medications, changing food selections in order to avoid undesirable interactions with certain taken medications or to avoid exceeding the upper limits of energy or nutrients, the need for engaging in physical activities, and to check for messages from his HCP 130, etc.

User Entries component 217 contains the various entries User 140 makes into the system described in the present invention; such as food and exercise selections, medications taken, current mood, any feeling of sickness, and location.

Indirect Inputs 220 is comprised of a set of inputs to the Engine Main Functions 200 other than those entered directly via UI 210. Example indirect inputs are given in the following sections.

Personal Information & Health History Records 221 contain information unique to each user and is used to personalize Advisor User Platform 100 and Secure Servers 110 parameters. Examples of the unique information are age, weight, height, gender, physical condition, chronic diseases, medications, allergies, and the like. This information may exist in Secure PHR 119, Secure Customer Records 114, Personal Usage History Store 104, and in EHR/EMR records maintained by HCPs 130.

Current Location 222 is the physical location User 140 is currently present at; such as home, restaurant A, restaurant B, school, etc. . . . . It is usually obtained from GPS Satellite 123 which is connected to Advisor User Platform 100 over GPS network 124.

Personal Health Device Measurements 223 are the measurements taken by User 140 as a result of using one or more Personal Health Device (PHD) 115. These measurements are usually uploaded to Advisor User Platform 100 over BAN/PAN/LAN network 121 as shown in FIG. 1, for eventual uploading to Secure Personal Health Records (PHR) 119 and Advisor Secure Servers 110.

User/HCP Adjustments 224 are updates typically made to the parameters of Advisor User Platform 100 or Advisor Secure Servers 110 by the User 140 or HCP 130 through accessing User Web Portal 141 and/or HCP Web Portal 131. More details will be discussed when describing step S311 in drawing FIG. 3 and an example is shown in FIG. 15, which will be described in the following sections.

FIG. 3 is a flowchart of the setup and initialization steps of the Advisor for a new User 140, which starts by invoking Start 5300 and continues to step S301 where secure remote access is established between User 140's Advisor User Platform 100 and Advisor Secure Servers 110 over LAN/WAN network 120.

The setup further entails establishing a new account for User 140 at Secure Advisor Servers 110 with the appropriate security credentials and saving encrypted copies of such security credentials in Personal Usage History Store's 104 non-volatile memory. The credentials allow User 140 to remotely access his health and medical information stored in Secure Customer Records 114 from mobile or stationary Advisor User Platforms 100's UI or from any other internet-connected device by invoking User Web Portal 141.

User 140 can also authorize other parties interested in monitoring his wellbeing such as Health Care Providers (HCP) 130 and relatives to access all or portions of his Secure Personal Records 114.

In step S302, Advisor User Platform 100 searches for and discovers all the Personal Health Devices (PHD) 115 available on BAN/PAN/LAN network 121. User 140 authorizes the PHD 115 devices to connect to one or more of the Advisor User Platforms 100 he intends to use for monitoring his health measurements generated by devices PHD 115.

As will be explained in detail in later sections, Advisor User Platforms 100 will also be used as bridges between PHD 115 and internet-connected systems and services; such as those hosted by Advisor Secure Servers 110 and PHR 119.

In step S303, System Initialization & Personalization function 201 creates secure communications links between Advisor User Platforms 100, Advisor Secure Servers 110 and User 140's Personal Health Records (PHD) 119 to allow the secure and private exchange of personal health information between these entities.

In step S304, User 140 authorizes one or more HCP 130 providers to securely access some or all information stored in his PHR 119 records to allow them to remotely monitor his health status and intervene, if necessary. This can be done by several methods, such as sending the designated HCP's 130 email messages with the required credentials and a link to a secure web site that processes the secure access requests.

Once HCP 130 has full or partial access to PHR 119, a link can be optionally established between HCP 130's EMR/EHR 118 electronic records system and PHR 119 to allow HCP 130 access to PHR 119 from his EMR/EHR 118 user interface.

In step S305, a secure 2-way audiovisual (A/V) communication link is established by System Initialization & Personalization function 201 between Advisor User Platform 100 and one or more HCP 130 designated remote sites. The secure 2-way A/V communications links allow User 140 to consult with one or more HCP 130 without the inconvenience of face-to-face visits.

In step S306, System Initialization & Personalization function 201 extracts personal information from the Personal Information & Health History Records 221.

Personal information extracted in step S306 is utilized by the Advisor to personalize User 140's nutrition and wellness advice and self-management system parameters. Examples of the personal information are age, gender, height, weight, ethnicity, chronic diseases, allergies, physical condition, handicaps, medications, home location, favorite foods, disliked foods, weekly food monetary budget, favorite restaurants, favorite grocery stores, favorite recipes, etc.

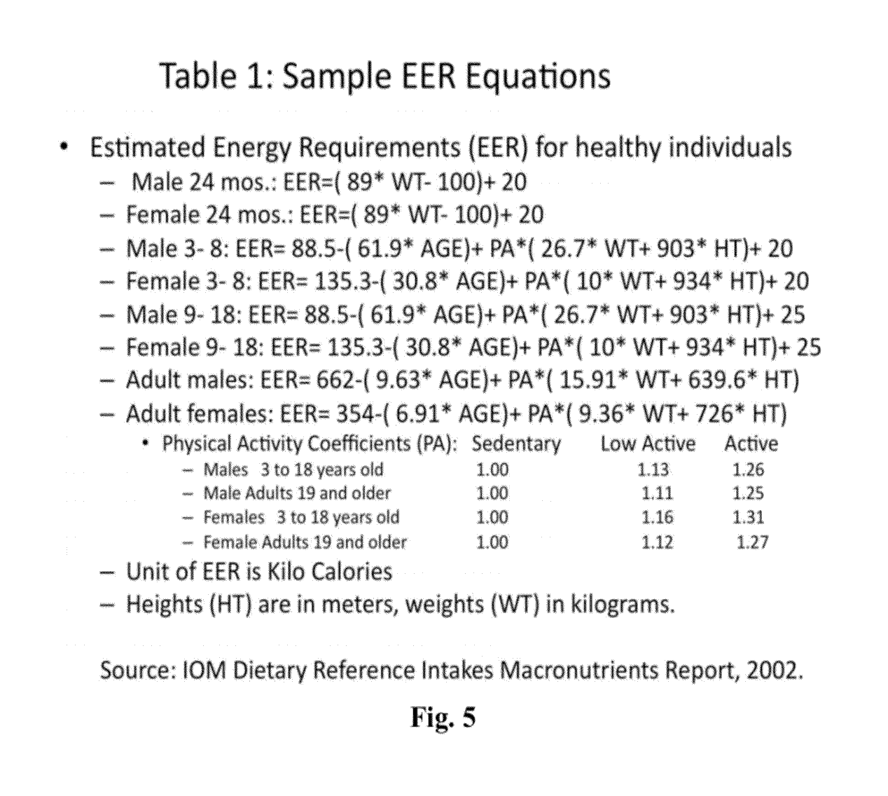

Utilizing User 140's personal information extracted in step S306, System Initialization & Personalization function 201 advances to step S307 where it selects the most appropriate equations and tables to be used for estimating User 140's daily energy and nutrient requirements. Sample equations and nutrient estimation tables are shown in Tables 1 through 6 presented in drawing FIGS. 5 through 10.

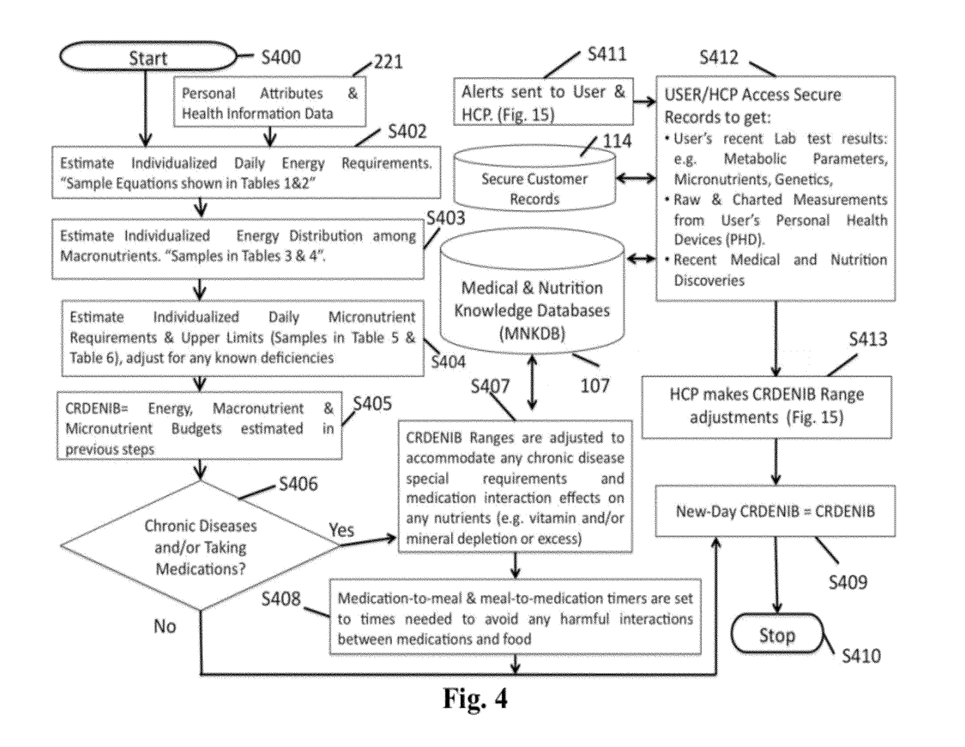

System Initialization & Personalization function 201 then advances to step S308, where it computes User 140's Initial Customized Required Daily Energy & Nutrient Intake Budgets (CRDENIB). The detailed computation steps of CRDENIB will be described in FIG. 4.

Once CRDENIB is computed in step S308, System Initialization & Personalization function 201 advances to step S309 where it creates the Initial Recommended Available Food Item List (RAFIL), which contains the food selections recommended for User 140's consumption.

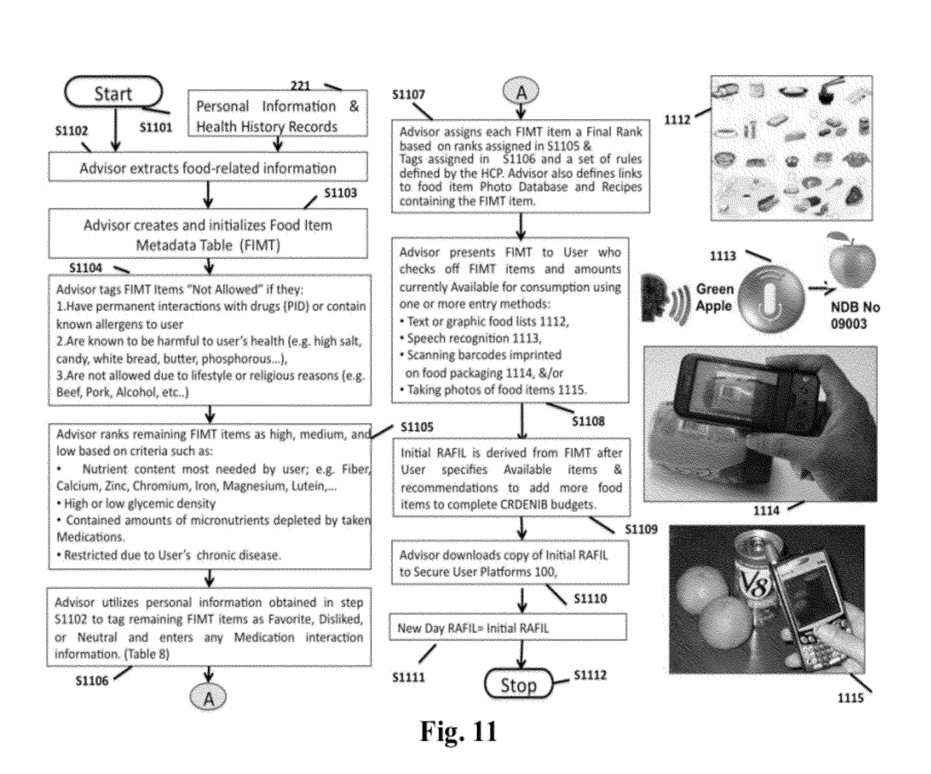

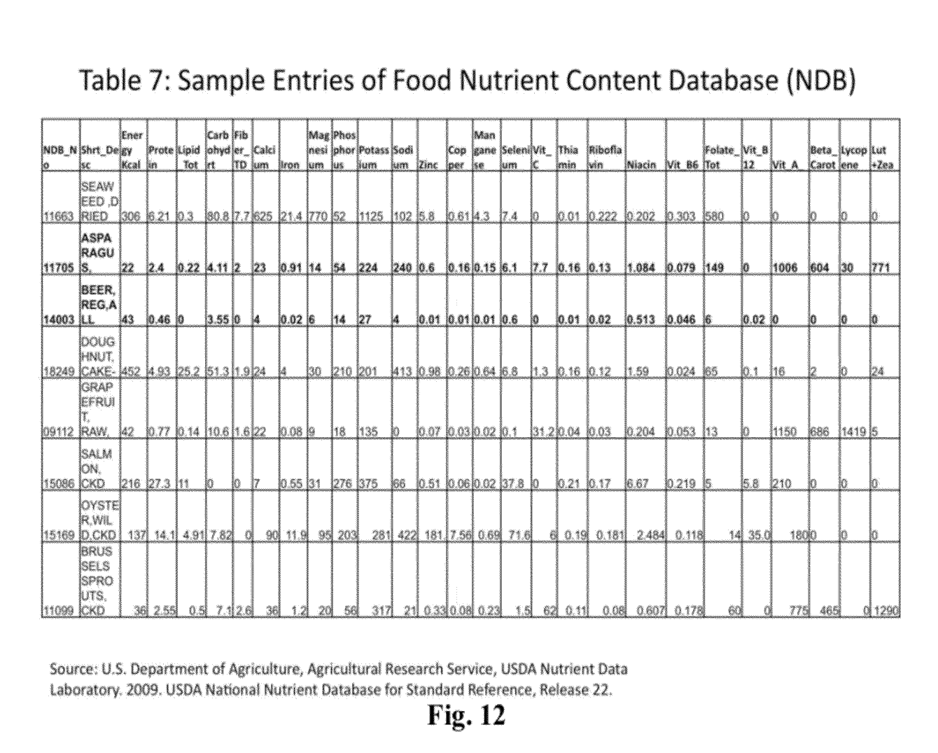

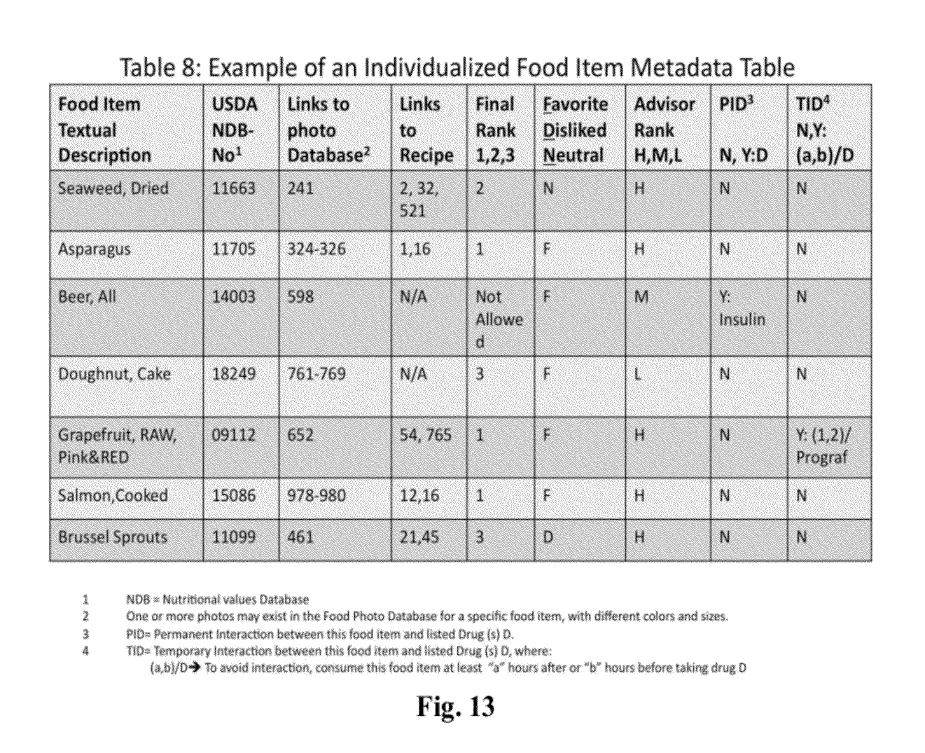

The details of the creation and updates of the RAFIL will be explained in more detail in FIG. 11 and the associated Table 7 and Table 8 presented in drawing FIGS. 12 and 13.

Once the initial RAFIL is created, System Initialization & Personalization function 201 then advances to step S310 where it designates the nutrition and wellness multimedia training materials most appropriate for User 140 based on his physical condition, chronic diseases, age, etc.

Links to the designated multimedia materials are downloaded to User 140's Advisor User Platform 100 for use by Personalized Chronic Disease, Nutrition & Wellness Training function 204 to present to User 140 during Interactive Multimedia Sessions 214.

Utilizing multimedia content expedites training users on how to utilize the features of the Personalized Nutrition and Wellness Advice and Self-management system and can be referenced and updated as frequently as needed and can be experienced on User 140's own schedule.

In step S311, System Initialization & Personalization function 201 initializes the timers and alarms for reminding User 140 to take his medications and to measure his health status indicators via Personal Health Devices 115 at the times and frequencies prescribed by HCP 130 providers. These timers are also utilized to synchronize the times medication and measurements are taken, with meal times and certain food types in order to avoid negative interactions between certain medications and certain foods and to make the measurements more accurate. This will be described more when describing drawing FIG. 16, “flowchart showing the operational steps of the Advisor during a typical day of use”.

In step S312, Advisor Secure Server 110 initializes Personal Health Device (PHD) 115 measurement charts based on the types of the PHD 115 devices discovered and authorized in step S302.

HCP 130 sets personalized normal ranges for each physiological parameter measurement, and alarms can be sent to User 140 and/or HCP 130 in case one or more measurements are significantly outside of the normal limits. The measurement charts are updated whenever new measurements are received from one or more PHD 115 devices.

Charting is done for measurements such as Blood Glucose, Blood Pressure, Weight, Medication consumption doses and timing, nutrient amounts consumed and consumption timing, physical activities performed, etc. . . . .

Copies of the charts are stored in User 140's Secure Customer Records 114, Secure Personal Health Records 119, and Personal Usage History Store 104, where User 140 and any designated HCP 130 providers and caregivers can view them to monitor User 140's health status.

These charts also enhance HCP 130 providers' ability to assess User 140's health status, conformance and response to prescribed medications and physical activities, as well as understanding User 140's self-management skills, eating habits and their effects on his general or specific health indicators.

For example, a 24-hour compound chart showing measurements and timings of User 140 Blood Glucose, Insulin and other medications taken, physical activities performed, and food intake allow HCP 130 to judge whether User 130's chronic diabetes mellitus is or is not under control, and can possibly show the causes for any out of normal range measurements.

HCP 130 can then take the appropriate intervention steps, such as changing medication types, doses, or timing, increasing or decreasing previously established energy and nutrient budgets (CRDENIB), adjusting food items in the RAFIL, or contacting User 130 to discuss reasons for not complying with the prescribed medications, physical activities, or diet.

If deemed necessary, HCP 130 can request User 140 to come to the office for a live checkup or can order additional lab tests. HCP 130 can also designate additional self-management training materials to be viewed by User 130, as described in step S310.

User 140 and any designated caregivers can also benefit from the measurement charts to increase their self-management skills by seeing first-hand the effects of diet, exercise, and missed medication doses on his health outcome. A pictorial example of the detailed intervention steps by User 140 and/or HCP 130 is shown in FIG. 15.

Alternatively, an expert system can be provided to make automatic health adjustment decisions and recommendations based on its knowledge of User 140's status and needs.

In step S313, System Initialization & Personalization function 201 initializes Personal Usage History Store 104, which holds local copies of User 140's favorite foods and their nutrient components, PHD measurement charts, medications, list of associated PHD 115 devices and their settings, HCP 130 communications, and other relevant logs of User 140's interactions with the Personalized Nutrition and Wellness Advice and Self-management system disclosed herein.

Finally, in step S314, User 140 is prompted to initialize his favorite locations and their geographical positions. Example locations are User 140's home, school, fitness club, favorite restaurants and grocery stores. This information, along with real-time GPS information obtained via GPS Satellite 123 will be utilized by Current User Location 222 to identify User 140's locations during the day and processed by Location-based Services function 205 to make the most appropriate nutrition and wellness recommendations for the present location.

For example, if it is detected that User 140 is currently at home, the food selection list (RAFIL) presented to him will only contain the recommended food items currently available at his home, or that can be ordered to be consumed at home.

Alternatively, if the Current User Location 222 indicates that User 140 is in restaurant A, the RAFIL presented to him will only contain food items available at restaurant A, which are most appropriate for User 140's best health outcome.

The initialization process ends by reaching step S315.

FIG. 4 is a Flowchart of the Recommended Daily Energy and Nutrient Intake Budget (RDENIB) Customization Process. The RDENIB customization process starts in step S400 and proceeds to step S402 where Personal Attributes & Health Information Data 221 is utilized to estimate User 140's estimated daily energy requirements.

Several equations for estimating a person's daily energy requirements are utilized by health care professionals, HCP 130, depending on User 140's personal attributes such as age, gender, height, weight, race, and health conditions such as chronic diseases and level of physical activity.

Table 1(Drawing FIG. 5) shows an example of a set of equations published by the International Organization of Medicine (IOM) Dietary Reference Intake Macronutrients Report 2002.

Assuming User 140 is a 32-year old female named Cindy weighing 60 kilograms with a height of 1.6 meters with a sedentary lifestyle (PA=1.0), we will use the sample equation for Adult Females, given in Table 1, to calculate Cindy's daily energy requirements:

EER=354−(6.91*AGE)+PA*(9.36*WT+726*HT)→Cindy's EER=1856 Kilo Calories

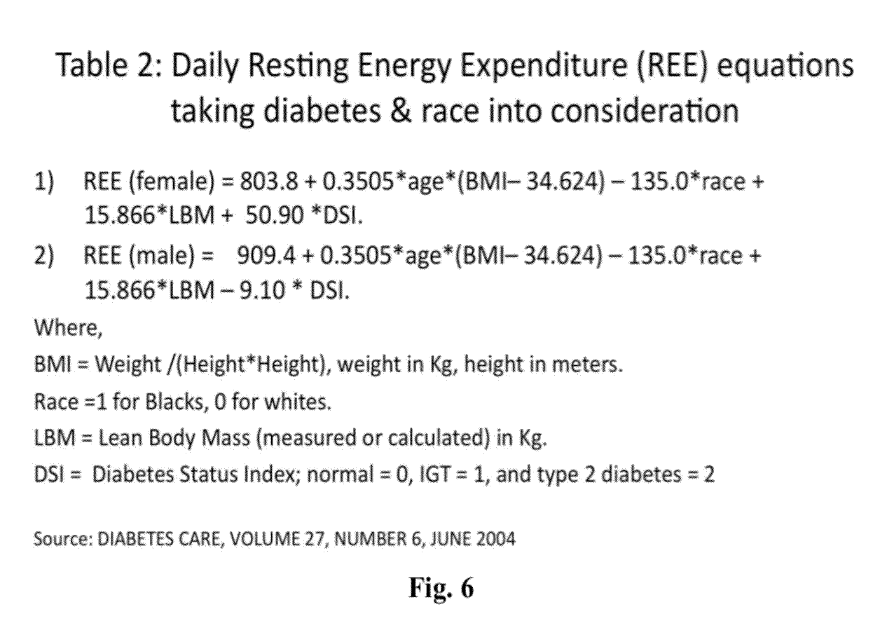

Now, assuming Cindy was a white Type 2 diabetic female, the system would use the equations shown in Table 2 (FIG. 6) to calculate her estimated resting energy expenditure:

REE(female)=803.8+0.3505*age*(BMI−34.624)−135.0*race+15.866*LBM+50.90*DSI→

Cindy's REE=1670 Kilo Calories; (with a calculated LBM of 43.4 Kg)

Once the estimated energy requirements for User 140 are calculated, the RDENIB customization process advances to step S403 where the distribution of User 140's energy requirements (energy budget) among the 3 major sources of energy (i.e. macronutrients), namely carbohydrates, protein, and fat is estimated.

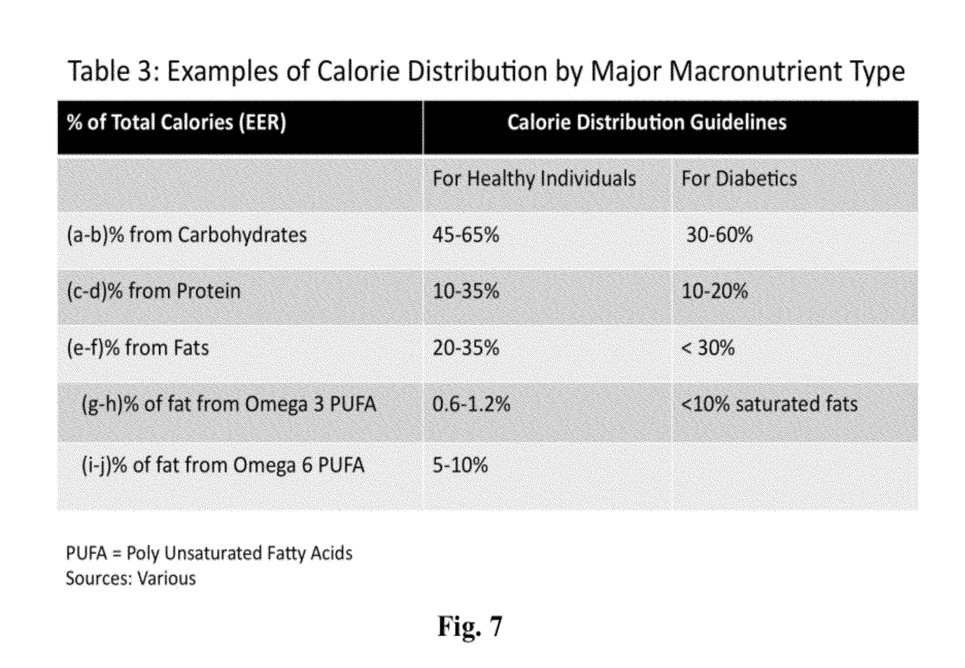

Table 3 (Drawing FIG. 7) shows an example of energy distribution ranges for two types of populations, healthy individuals and diabetics.

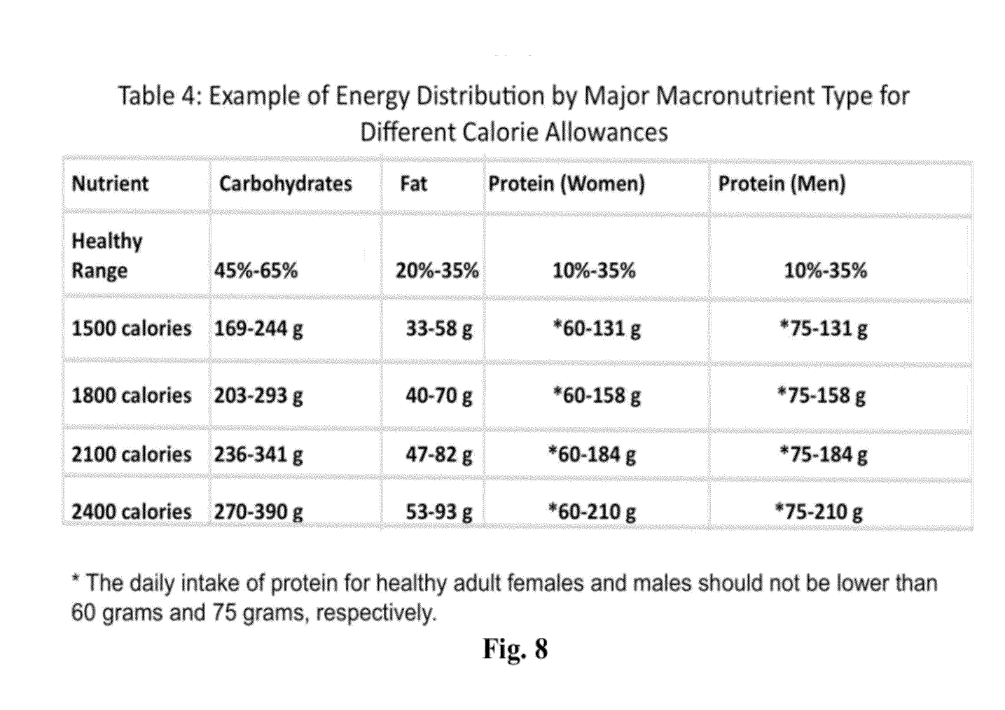

Table 4 (drawing FIG. 8) shows another example of an energy (i.e. Calorie) distribution range guideline shown for certain estimated energy requirements values. The distribution ranges are shown in terms of grams, where 1 gram of carbohydrates and protein contains 4 Kilo Calories each and 1 gram of fats contains 9 Kilo Calories.

Knowing that User 140 Cindy is diabetic with an estimated REE of 1670 Kilo Calories, the system would use the following macronutrient energy distribution given in Table 3: 30-60% of Cindy's 1670 calories (501-1002 Kilo Calories) should come from consumed carbohydrates, 10-20% (167-334 Kilo Calories) should come from consumed Protein, and less than 30% (less than 501 Kilo Calories) should come from consumed fat, with less than 10% of fat calories (less than 50 Kilo Calories) to come from saturated fats.

These ranges and values are just examples used here to demonstrate the operation of an embodiment of the present invention. During actual practice of the present invention, HCP 130 will estimate the most appropriate energy distribution values for User 140 and store them in User 140's CRDENIB variable.

Once the energy distribution ranges for User 140 are estimated, the RDENIB customization process advances to step S404 where it estimates User 140's individualized daily micronutrient requirements & Upper Limits.

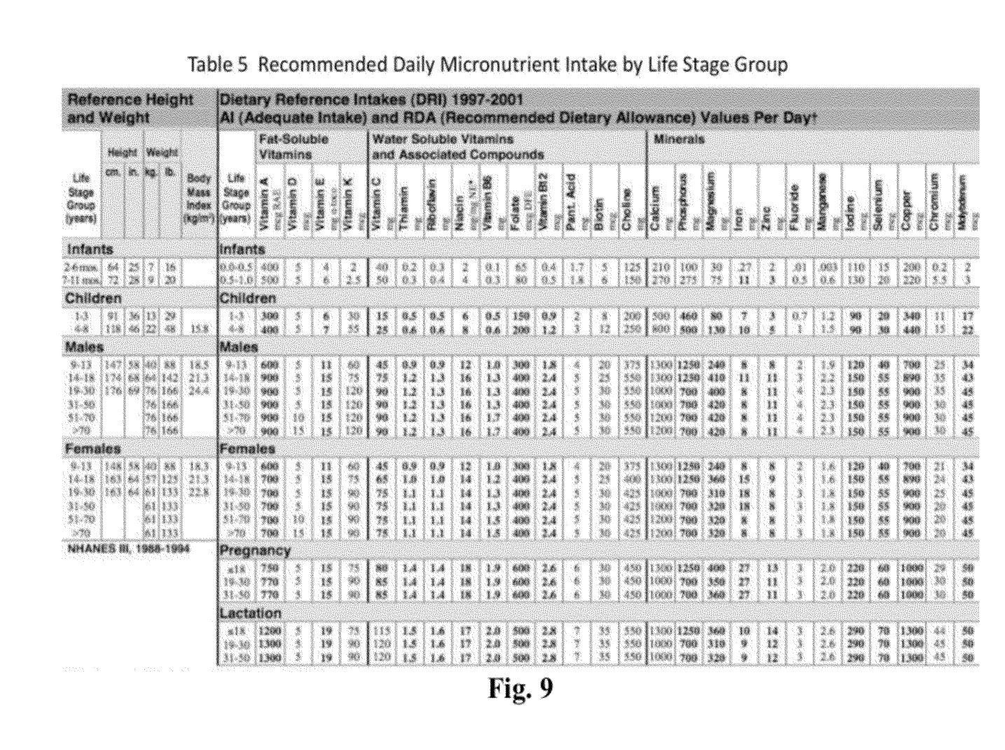

Table 5 (drawing FIG. 9) shows an example of recommended daily micronutrient intake values for a variety of life stage groups, such as infants (2 age groups), children (2 age groups), males (6 age groups), females (6 age groups), pregnant females and lactating females (3 age groups).

These recommendations are just examples used to demonstrate the operation of an embodiment of the present invention. During actual practice of the present invention, HCP 130 will determine the most appropriate values of micronutrients to be recommended for consumption by User 140 for his best health outcome.

For User 140 Cindy, the values assigned to Females age 31-50 will be used to find the recommended daily values of micronutrients (vitamins and minerals). Example of some micronutrient values extracted from Table 5 for Cindy are:

Vitamin A: 700 micrograms

Vitamin D: 5 micrograms

Vitamin C: 75 milligram

Niacin: 14 milligrams

Vitamin B6: 1.3 milligrams

Magnesium: 320 milligram

Zinc: 8 milligrams

Step S404 also estimates the Upper Limits for each of the micronutrients, which User 140 is advised to not exceed in order to avoid harmful effects. This information is available to healthcare providers in the medical literature and the Upper Limit values vary from person to person.

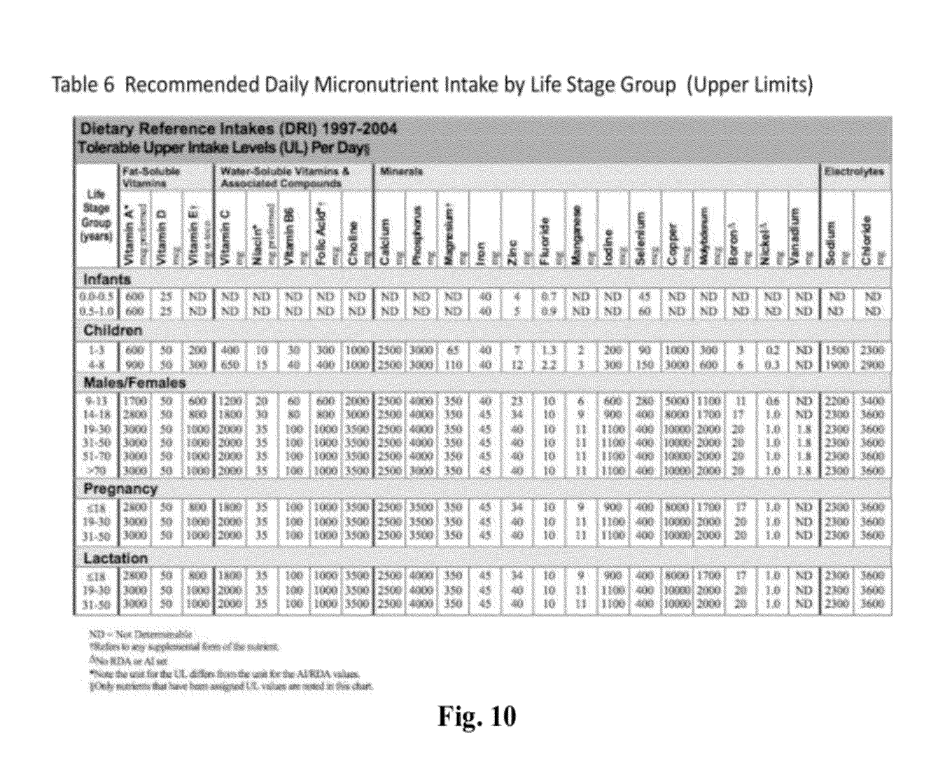

Table 6 (drawing FIG. 10) provides an example of the Upper Limits for many vitamins, minerals, and electrolytes based on users' life stages; infants, children, males/females ages 9 to >70 years, pregnant, and lactating females.

These values are just an example used to demonstrate the operation of the present invention. During actual practice of the present invention, HCP 130 will determine the most appropriate values of micronutrients' Upper Limits to be recommended for “do not exceed” by User 140 in order to avoid health complications.

Referring to our User 140 Cindy again, the Upper Limits values in row 31-50 under Males/Females will be used. Some Upper Limit values are:

Vitamin A: 5000 micrograms

Vitamin D: 50 micrograms

Vitamin C: 2000 milligrams

Niacin: 35 milligrams

Vitamin B6: 100 milligrams

Magnesium: 350 milligrams

Zinc: 40 milligrams

The upper limits for certain nutrients may be increased beyond the published upper limits for healthy individuals for some users with known micronutrient deficiencies, which are usually detected by performing a specific or comprehensive micronutrient test in certified laboratories.

Step S405 establishes User 140's customized recommended energy and nutrient budget (CRDENIB), from the values of total energy, energy distribution among macronutrients, and recommended and Upper Limit values of micronutrients, which were estimated in steps S402, S403, and S404.

Using the estimations made above, User 140 Cindy CRDENIB budgets look like this:

Total Energy budget=1670 Kilo Calories

Range of calories from consumed Carbohydrates=501-1002 Kilo Calories

Range of calories from consumed Protein=167-334 Kilo Calories

Calories from consumed fats<501 Kilo Calories

Calories from saturated fats<50 Kilo Calories

Micronutrient ranges:
Vitamin A=700-5000 micrograms
Vitamin D=5-50 micrograms
Vitamin C=75-2000 milligrams
Niacin: 14-35 milligrams
Vitamin B6: 1.3-100 milligrams
Magnesium: 320-350 milligrams
Zinc=8-40 milligrams

In step S406, the CRDENIB customization function checks User 140's Personal Attributes & Health Information Data 221 to see if User 140 has any chronic diseases and/or takes any medications. If the answer is No, flow continues to step S409 where the variable “New-Day CRDENIB” is set to the values established in step S405. New-Day CRDENIB is the energy and nutrient budget allocated to User 140 at the beginning of the day. The initial CRDENIB customization process is now complete and it stops in step S410.

If, however, the answer to the question in step S406 is Yes, meaning that User 140 has at least one chronic disease or is taking one or more medications, flow is transferred to step S407 where Medical & Nutrition Knowledge Databases 107 are consulted for any possible effects User 140's one or more chronic diseases and/or one or more medications may have on any of the recommended energy and nutrient values.

The CRDENIB ranges are then adjusted to the special nutrient needs User 140's chronic disease(s) may impose. For example, it is known that patients with chronic cardiac disease should reduce their intake of sodium and increase their intake of potassium. It is also known that diabetes mellitus type 2 patients require higher levels of the mineral magnesium than people who are not diabetic. There is also evidence that higher dosages of Niacin may delay kidney damage in diabetics. Since User 140 Cindy is diabetic, the Advisor consults with Medical and Nutrition Knowledge Databases (MNKDB) 107 which recommend that Cindy's Magnesium and Niacin intake ranges be increased to:

Magnesium: 400-600 milligrams per day

Niacin: 25-50 mg/kg/day 4 150-300 mg/day (Cindy weights 60 Kg)

Cindy's updated CRDENIB ranges are now:
Total Energy budget=1670 Kilo Calories
   Range of calories from consumed Carbohydrates=501-1002 K Calories
   Range of calories from consumed Protein=167-334 Kilo Calories
   Calories from consumed fats<501 Kilo Calories
      Calories from saturated fats<50 Kilo Calories
Micronutrient ranges:
   Vitamin A=700-5000 micrograms
   Vitamin D=5-50 micrograms
   Vitamin C=75-2000 milligrams
   Niacin: 150-300 mg
   Vitamin B6: 1.3-100 milligrams
   Magnesium: 400-600 milligrams
   Zinc=8-40 milligrams

Although we will not show all the adjusted CRDENIB values in this disclosure, CRDENIB values of other minerals and vitamins such as Vitamin C, B12, Thiamin, Chromium, and Zinc are also likely to be adjusted for diabetics in step S407.

Similar to what was shown above for a diabetic patient, adjustments to vitamin and mineral recommended values would also be made for patients with heart disease, cancer, and other chronic diseases.

Step S407 will also compensate for any negative medication effects on certain nutrients or positive effects of certain nutrients on the absorption of medications. This is done by increasing or decreasing the values of these nutrients in CRDENIB.

For example, there is evidence that the popular diabetes mellitus medication Glucophage (Metformin) reduces the mineral folate. Folate may prevent retinopathy and nephropathy, which are serious long-term effects of diabetes. To compensate for the loss of this essential micronutrient, the Advisor system disclosed herein will consult with commercially available MNKDBs 107 to find out the appropriate increase in the recommended amounts of Folate for patients taking the medication Glucophage.

There is also evidence that magnesium helps in the efficient utilization of insulin in lowering blood glucose.

On the other hands, some chronic diseases such as chronic kidney failure, require that the intake of certain minerals, such as phosphorous be consumed in reduced amounts. In these cases, step S407 will discover this information by consulting MNKDB 107 and will reduce User 140's Upper Limit intake of phosphorous accordingly.

In step S408, based on information obtained from MNKDB 107, medication-to-meal & meal-to-medication timers are set to times needed to avoid any harmful interactions between medications and nutrients in certain foods.

Once the appropriate adjustments to nutrient values are made, control moves to step S409, where the variable New-Day CRDENIB is set to the adjusted values. The initial CRDENIB customization process is now complete and it stops in step S410.

In addition to taking into account chronic diseases and medications to adjust CRDENIB values, the CRDENIB values are also likely to be adjusted during the practice of the present invention when certain asynchronous events and activities take place. Example events are given below.

In those cases, step S411 shows that alerts will be sent to User 140 and HCP 130 by the Advisor system so they can take action to adjust the CRDENIB values to compensate for the effects of the various reported events.

In step S412, upon receiving an alert, HCP 130 and/or User 140 access User 140's secure records 118 and 119 to observe the events that led to generating the alarm. Three examples of such events are listed below.

Event 1: User 140's recent laboratory test results show deficiencies, or excesses, in certain vitamins and nutrients. These tests could have been routine tests for metabolic parameters, or could have been requested by HCP 130 to check certain micronutrient levels due to particular symptoms User 140 has complained about, or may have been done as a proactive measure to predict certain genetic predispositions to chronic diseases which may be preventable by proper nutrition.

Event 2: Abnormal measurement trends obtained from heath status measurements taken by User 140 via Personal Health Devices 115 and charted by Health-related Measurement Monitoring & Charting Services 206. A flow diagram of the steps taken during the PHD 115 measurement reporting and corresponding HCP 130 actions is shown in FIG. 15.

Event 3: HCP 130 may also adjust User 140's CRDENIB values based on recent discoveries published in professional journals or in Medical & Nutrition Knowledge Databases 107.

In step S413, HCP 130 makes the necessary adjustments to User 140's CRDENIB values in response to the problems or new information discovered in step S412. More details on these adjustments are shown in FIG. 15 and will be described in later sections.

Control then moves to step S409 where the variable New-Day CRDENIB is set to the updated CRDENIB values.

The CRDENIB adjustment process is now complete and it stops in step S410.

Now that the CRDENIB has been established to meet User 140's macronutrient and micronutrient daily requirements for maintaining optimum health outcomes, the next step in initializing the Personalized Nutrition and Wellness Advice and Self-management method and system (Advisor) is to construct a personalized Recommended and Available Food Item List, RAFIL, mentioned earlier when describing step S309.

The contents of the RAFIL will be dynamically varied by the inventive Advisor method and system to assist User 140 in consuming the appropriate portions and variety of foods containing the energy and nutrient amounts specified in User 140's Customized Recommended Daily Energy and Nutrient Intake Budgets, CRDENIB.

Drawing FIG. 11 shows the steps taken by one example embodiment of the Advisor method and system to create an initial RAFIL personalized for User 140.

The RAFIL creation process starts at step S1101 and proceeds to step S1102 where the food-related information relevant to User 140 is extracted from Personal Information & Health History Records 221. This is information such as favorite & disliked foods, disallowed foods for religious or lifestyle reasons, food allergies, weekly food monetary budget, chronic diseases, favorite restaurants, favorite grocery stores, medications taken, etc. . . . .

The RAFIL creation process then advances to step S1103, where the Advisor creates and initializes a Food Item Metadata Table (FIMT). Initially, the FIMT has one entry for each unique food item contained in the one or more Food Nutrient Content Databases 108 utilized by the present invention. One example food nutrient database is the USDA National Nutrient Database for Standard Reference, Release 22; a portion of which is shown in Table 7, FIG. 12. As will be described later, the FIMT will have additional entries for custom food items entered by User 140 and/or HCP 130, which do not exist in the standard Food Nutrient Databases 108.

Each FIMT entry has several attributes (metadata) identifying and describing the evaluation results of each food item. Examples of the attributes are shown in the top raw of Table 8 (FIG. 13) are listed in the following sections.

Nutrition Database index number (e.g. USDA database NDB-No) which is a unique pointer to the food item's energy and nutrient content information stored in Databases 108,

Links to Food Photo Database where photographs of food items are stored. Food item photographs will be used in at least a couple of situations: For entry of food items into the RAFIL as will be explained in step S1108, and for presentation to User 140 to make his food selections; as a more friendly method to showing a textual representation of the available food item list.

Links to Recipes, which use the food item as an ingredient and will be recommended to User 140 to motivate him into consuming healthy food items. Sample recipes are stored in the Advisor system's Educational & Motivational Multimedia Content Library 109,

Final Rank, which will be defined in step S1107.

User 140's vote on the food item: Favorite, dislike, and neutral. More will be described in step S1106.

Advisor ranking of each food item as explained in step S1105.

Permanent Interaction with Drugs (PID) assessment as determined by utilizing the personal information extracted in step S1102, in addition to consulting with Medical and Nutrition Knowledge database 107.

Temporary Interaction with Drugs (TID) assessment as determined by utilizing the personal information extracted in step S1102, in addition to consulting with Medical and Nutrition Knowledge database 107.

The FIMT will be populated in subsequent steps with various attributes that will be used in the RAFIL construction and presentation decision-making procedures and in the process of generating medication timing alerts, to be described in later sections.

The RAFIL creation process then moves to step S1104, where the Advisor scans all items in a designated Food Nutrient Content & Physical Activity Database 108 and tags Food Items “Un-allowed” in the corresponding FIMT entry. Example Unallowed food items are those which have Permanent Interactions with Drugs (PID) or contain known allergens to user; are known to be harmful to user's health (e.g. high salt, candy, white bread, butter, phosphorous for kidney failure patients . . . ); are not allowed due to lifestyle or religious reasons (e.g. Beef, Pork, Alcohol, etc.), or that User 140 indicated that he will never eat or drink.

An example is shown in Table 8 (Drawing FIG. 13) where the food item Beer is tagged un-allowed due to its undesired interactions with the drug Insulin. Based on this tagging, Beer will not be presented to the User 140 in any future RAFIL's.

The advantage of tagging food items in the FIMT as un-allowed is that it would minimize the size of the FIMT leading to a significant reduction in speed during the database search operations done throughout the practice of the present invention; as will become clearer when explaining the operation of the Advisor in FIGS. 15 & 16.

The next step in the RAFIL creation process is S1105, where the RAFIL creation function of the Advisor system ranks the remaining food items in Table 8 as high, medium, or low based on certain criteria.

For example, High ranks may be assigned to food items which are high in nutrients needed most by user; e.g. Fiber, antioxidants, calcium, zinc, chromium, iron, Omega 3 fatty acids, magnesium, lutein, folate, etc.

Another example is when High ranks may be assigned to food items high in micronutrients depleted by medications taken by User 140. For example, foods high in magnesium are ranked high when User 140 is taking the glucose-lowering medication Metformin, which is known to deplete the mineral magnesium.

In another example, Low ranks may be assigned to food items with high glycemic density if User 140 is diabetic and items with Low glycemic density, which are better for diabetics, will get higher rankings

Low ranks will also be given to food items high in micronutrients, which can be harmful to User 140, such as Phosphorous for users with kidney failure.

After the Advisor completes ranking all entries of the food nutrient database based on their nutrient contents, it moves to step S1106 where the Advisor utilizes personal information obtained in step S1102 to tag the remaining food items as Favorite, Disliked, or Neutral (meaning User 140 does not mind eating, but is not a favorite food).

Any medication interaction information with each food item is indicated in the attributes PID and TID, as shown in Table 8, where Beer was discovered to interfere negatively with the medication (drug) insulin and was noted in attribute PID. Another example is Grapefruit, which was found to interact temporarily with the drug Prograf if both were taken too close in time. Grapefruit's attribute TID was assigned the values (1,2), Prograf. This tells the Advisor system that User 140 should be alerted if he attempts to eat grapefruit less than 2 hours before the scheduled time of taking Prograf or less than 1 hour after taking Prograf.

In step S1107, the Advisor assigns each FIMT item a Final Rank based on ranks assigned in S1105 & Tags assigned in S1106 and a set of rules defined by the HCP 130. For each entry of the FIMT, the Advisor also defines links to food item Photo Database and Recipes containing one or more photos of the FIMT item. The food item photos can be used for one of two purposes. The first purpose is to display the photos of food items in the RAFIL as a more user-friendly way than showing the RAFIL is text format. The second purpose for having the food items represented by photos is to enable the recognition of food items entered into the RAFIL, as will be explained in later steps, during the process of constructing and maintaining the varying versions of RAFILs.

The Final ranks are assigned as follows. Highest final ranks (1) are given to items ranked High by Advisor & favored by user. Average final ranks (2) are given to food items with High Advisor ranks and Neutral user preferences. Lowest final ranks (3) are given to items ranked low by Advisor and disliked by user.

Table 8 shows a sample of a few entries in a filled out Food Item Metadata Table, FIMT.

Although the first item, Seaweed, is given a High rank by the Advisor due to its high content of such nutrients such as Magnesium, Potassium, and Folate, it scores a Final Rank of 2 due to User 140's indication that it is not a favorite food, but he did not mind eating it (neutral).

The food items Asparagus, Grapefruit, and Salmon are assigned a final Rank of 1 because they received a High Advisor ranking and are Favorite foods of User 140.

The food item Doughnut is assigned a Final Rank of 3 although it is designated a Favorite of User 140. This is due to Advisor ranking of Low due to its high Glycemic index and very poor content of micronutrients.

Finally, the item Brussels Sprouts is assigned a Final Rank of 3 because it is tagged “Disliked” by User 140, regardless of its rich micronutrient content.

The ranking criteria described above are just examples to demonstrate the ability of the present Advisor method and system to personalize food item recommendations for each user that are both nutritious and favored by each specific user while accommodating any chronic diseases and medication schedules.

Additionally, the food item personalization has the advantage of increasing the chances that users will consume healthy amounts of nutrients, without exceeding the energy budgets as will be explained later in FIGS. 15, 16A, 16B, and 17.

During practice of the disclosed Advisor method and system described herein, healthcare professionals will be able to customize ranking criteria further with the assistance of commercially available Medicine and Nutrition Knowledge databases, such as MNKDB 107.

In step S1108, the Advisor presents the FIMT to User 140 who checks off all FIMT items, which are currently “Available” for consumption using one or more entry methods described below.

The preferred food item selection/entry method is shown in Available Food Item Entry Method #1, shown is diagram 1112, and is comprised of selecting food items to enter into the personalized food item library from a text or graphical list showing a multitude of various food items in different categories; such as meats, dairy, fruits, vegetables, grains, User 140 favorite restaurants' food menu items, items from User 140's favorite grocery store, etc.

Another method is Available Food Item Entry Method #2, shown in diagram 1113. Using speech recognition technology in Method #2, User 140 utters the description of the food item he wishes to enter into the food library. The speech recognition engine supported by Advisor User Platform 100 will translate the speech uttered by User 140 into a graphical or textual representation of the food item. User 140 confirms the item and it gets stored into the food item library.

A third method is Available Food Item Entry Method #3, shown in diagram 1114, and is comprised of scanning a bar code label imprinted by the manufacturer on the food item. The bar code is translated into a unique product code, which is sent to a database, which translates the bar code into a description of the food item and a list of its major nutritional components.

A fourth method is Available Food Item Entry Method #4, shown in diagram 1115, and is comprised of taking photographs of the selected food item(s). The photographs can be then analyzed and translated into the particular food items names and food database addresses (e.g. NDB_No) and portions by known image recognition methods not relevant to this invention.

Control advances to step S1109, where the Advisor system and method derive the Initial Recommended Available Food Item List (Initial RAFIL) from the information stored in the completed FIMT in a few steps described below.

The first step is to extract the names of all FIMT food items that have been checked off by User 140 in step S1108 as “Available”.

The next step is to evaluate whether the available food items contain all the required nutrients (both macronutrients and micronutrients) necessary to satisfy User 140's requirements defined in the steps contained in FIG. 4 and described earlier.

One method to accomplish this evaluation is by adding all nutrients contained in all Available food items for x numbers of days and comparing the result with the user's CRDENIB budgets for x days. If the Available food items contain all the CRDENIB components, no further action is needed. If however, it is determined that some nutrient budget goals will not be met by consuming the Available food items, the system will recommend adding more food items to the RAFIL which are rich in the inadequate nutrients.

One source of these food items would be User 140's favorite supermarket/grocery store indicated in the Personal Information & Health History Records 221. The system accesses the supermarket's web site and retrieves the names of the recommended food items and presents them as a shopping list for User 140 to purchase. Alternatively, the desired food items may be purchased on-line and delivered to User 140's home. After User 140 purchases the new food items, they will be entered in the system as was described in step S1108.

Finally, control moves to step S1110 where the Advisor downloads a copy of the Initial RAFIL to one or more of User 140's Advisor User Platforms 100, where it can be presented in textual or graphic format, depending on each Advisor User Platform 100 display capabilities and user preference.

The actual subset of the RAFIL food items presented to a particular User 140 at any given point in time will depend on many factors, such as remaining CRDENIB budget, location, time of day, medication type and timing, and food item Final Ranks. The details of RAFIL usage and updates will be explained in more details in FIGS. 16A and 16B.

The Secure User Platform 100 variable New Day RAFIL is set to the contents of the Initial RAFIL in step S1111 and the RAFIL initialization function completes execution and stops at step S1112.

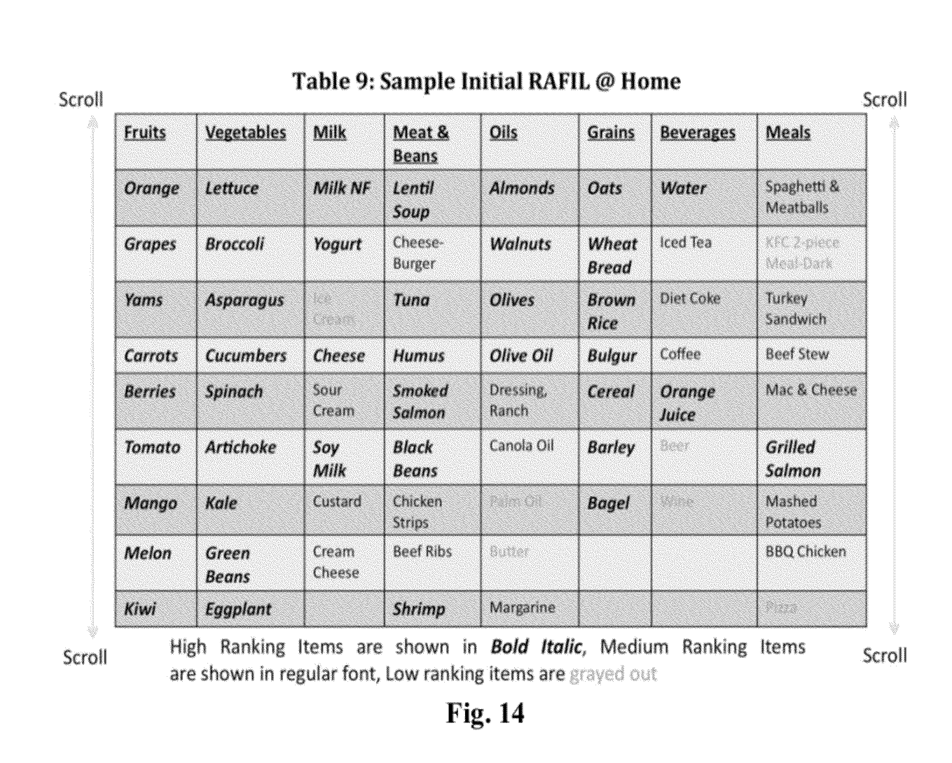

A Sample Initial RAFIL in textual format is shown in Table 9 (FIG. 14). Each column in Table 9 shows a different food group with the recommended food items User 140 will select his meals and snacks from. In this example, the 6 food categories recommended by the USDA are shown; Fruits, Vegetables, Milk, Meat & Beans, Oils, and Grains. I added Beverages to manage and keep track of fluid intake and a Meals column to present complex food items and accommodate Restaurant meals.

As can be seen in Table 9, the food items in this particular RAFIL are currently available for consumption at User 140's home. The food items ranked High by the Advisor are presented to User 140 in Bold Italic font, the Middle ranks are presented in Regular font and the Low-ranked items are presented in Grey font. Alternatively, the RAFIL can be presented in a graphic format where photos of the food items are shown on Advisor User Platform 100's screen.

After User 140's daily energy and nutrient budgets (CRDENIB) and the initial recommended available food item list, RAFIL, have been initialized, these and other Advisor system variables will get updated throughout the day due to various activities and events and to keep all energy and nutrient budgets balanced.

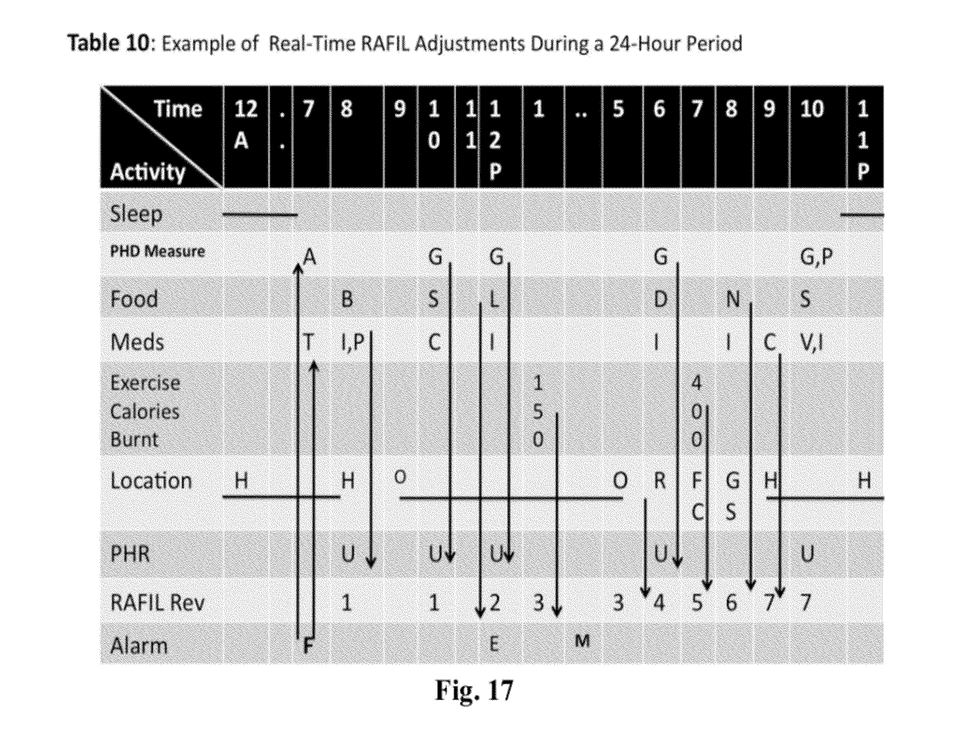

Examples of these adjustments and balancing actions will be shown in FIGS. 15, 16A, and 16B with the assistance of the examples given in Tables 10, 11, 12, and 13 (FIGS. 17, 18, 19, and 20).

FIG. 15 shows an example of how the PHD 115 measurements routinely taken by User 140 are utilized to cause adjustments to be made to Advisor system variables, such as CRDENIB, RAFIL, medication management plan, and physical activity plan.

In reaction to the new information the Advisor system learns about User 140's health status, through the measurements contained in PHD devices 115 and communicated over various networks, the Advisor system can adjust some non-critical variables automatically; such as increasing the energy budget after exercising to compensate for the energy lost during the exercise activities.

When HCP 130 analyzes these measurements he may adjust all critical variables, such as medication types and dosages; such adjustments can be done remotely as described earlier by accessing and updating the relevant variables stored in User 140's Personal Health Records 118 and Electronic Health Records 119.

User 140 himself may be authorized to change some less critical variables, such as indirect changes to RAFIL through acquiring more healthy food items, which the Advisor system will analyze for nutritional content and rank them accordingly, as previously explained in FIG. 11's description.

Step S1501 shows User 140 routinely taking measurements of his health status utilizing Personal Health Devices 115. Some measurements are taken multiple times per day, such as blood glucose measurements taken before and after meals, confirmations of taken medications from a Medication Monitor, or the measurements are taken once per day, such as getting weighed on a weight scale every morning.

These measurements are uploaded in Step S1502 to one or more of User 140's Advisor User Platforms 100, over a wired or wireless network such as BAN/PAN/LAN network 121. The Advisor User Platform 100 acknowledges receipt of the PHD measurements and stores a copy in User 140's Personal Usage History Store 104.

Next, in step S1503, Advisor User Platform 100 either batches the set of User 140 physiological measurements and uploads them to the Advisor Secure Servers 110 at set periods of time (e.g. every 30 minutes) or alternatively it can be instructed to check the measurements for any out of limit values and if any out of limit measurements are detected, the measurements are immediately uploaded to Advisor Secure Servers 110.

In step S1504, the Advisor Secure Server compiles & charts one or more of User 140's sets of measurement data points and sends a copy to User 140's Secure Customer Records 114 and another copy to User 140's Personal Health Record 119.

In step S1505, alerts are sent to User 140 & HCP 130 to inform them that new data regarding User 140's health measurements is available for viewing. The alerts could be sent via email, SMS, voice call, or any other communications method. The urgency of the alert will depend on the seriousness of User 140's physiological parameter measurement data.

In step S1506, HCP 130 responds to the alert message by accessing the authorized section of User 140's PHR 119 where the measurement charts are stored. HCP 130 then analyzes the PHD 115 measurement trends & recent lab results stored in User's PHR 119.

In step S1507, HCP 130 may check the latest released medical and nutrition information in Knowledge Bases 107 for advice on the best adjustments to be made to User 140's health plan.

Based on the results of HCP 130's analysis, in step S1508, HCP 130 makes any necessary adjustments to User's RDENIB, food item lists (RAFIL), medications, multimedia training materials, and/or Physical Activity plan & saves all adjustments to User's Secure Customer Record 114 &/or PHR 119.

The Advisor system detects that HCP 130 made some adjustments, and control is transferred to step S1509 where the Server downloads the various nutrition and wellness parameter adjustments through User Advisor Platform 100 indirect input port 224 as described earlier in step S413.

Finally, in step S1510, User 140 is notified of any adjustments made to his nutrition and wellness plans. In case the reported results require additional lab work to be performed, or face-to-face discussions between User 140 and his HCP 130, User 140 will be informed to make the required appointments to get the required tests and consult with the HCP 130.

FIGS. 16A and 16B, along with the examples shown in FIGS. 17, 18, 19, and 20, show the detailed user experience and processing steps taken by the Advisor system during a typical day (24 hours) of User 140's usage of the disclosed Advisor system.

For Table 10 (FIG. 17), arrows indicate an event which causes change (s) to RAFILs or signifies that information is uploaded to PHR or an Alarm/Reminder sent to User or HCP, A=All Tests, G=Glucose, P=Pressure, B=Breakfast, L=Lunch, D=Dinner, S=Snack, H=Home, O=Office, R=Restaurant, FC=Fitness Center, E=Exercise, F=T on empty stomach/Test All, U=Upload, I=Insulin, C=Cholesterol, V=Supplement, T=Thyroid, M=Meds OK, GS=Grocery Store, N=New Food.

The various steps described below demonstrate the Advisor system and methods' effectiveness in ensuring that User 140 attains an optimized health outcome by:

Getting the personalized requirements of nutrients (macronutrients and micronutrients) while consuming his favorite foods (when possible) at home or at restaurants;

not exceeding the maximum allowed amounts of energy and nutrients;

performing sufficient customized physical activities;

accommodating sick days by varying the recommended food items;

taking his medications safely and timely, and

getting timely and accurate interventions from his healthcare providers in response to his daily heath status measurements.

Referring to FIG. 16A, processing starts when step S1600 is entered. This typically occurs when Advisor User Platform 100 is turned on at the beginning of a new day. Control moves to step S1601 where the CRDENIB and RAFIL variables are reset to the New Day values, “NEW DAY CRDENIB” and “NEW DAY RAFIL” respectively, as was described in detail earlier.

The Advisor system then moves to step S1602 where it sends reminders to User 140 to take any medications that must be taken on an empty stomach and sets reminders for the rest of the day to take other medications with food, or plenty of water, if so recommended by the drug manufacturers. These are important reminders in order for User 140 to realize the best therapeutic effects of such medications while preventing harmful side effects. An example of a medication reminder issued by the Advisor system is shown in Table 10 (FIG. 17) where an Alarm F is sent at 7 AM to remind User 140 to take his T (Thyroid) medication on an empty stomach.

Alarm F can be also programmed to remind User 140 to utilize Personal Health Devices (PHD) 115 to take any health status measurements, such as blood glucose, weight, blood pressure, temperature, etc.

Alarms are also set in step S1602 to remind User 140 to perform the required physical and mental exercises.

The next step is S1603 where the Advisor user interface asks User 140 if he plans to eat now; (alternatively User 140 can press a designated button on Advisor User Platform 100's User Interface 210 block 217, when he is planning to eat). If User 140 responds with a negative answer of NO, control advances to step S1604 where User 140 is asked if he has just exercised, or if he would like recommendations on certain exercises. If User 140 responds with a negative answer of No, control will be advanced to step S1606 where User 140 is asked if he has recently acquired additional food items (e.g., from Grocery Store, brought by friends or visitors, etc. . . . ). If the answer to question S1606 is No, control returns to step S1603 where the Advisor will wait for User 140's next interaction.

However, if User 140 has indeed acquired new food items (such event is shown in Table 10 (FIG. 17) where User 140 visits a Grocery Store, GS, around 8 PM, and purchases new food items, marked in the Food row as N), control will move to step S1607 where User 140 enters the new food items into Advisor Platform 100 utilizing one or more of the input methods described previously in step S1108.

As a result of adding the newly acquired food items, the RAFIL selections and rankings are adjusted. Table 10 (FIG. 17) shows event N causing a new RAFIL Revision 6 to be generated based on the addition of new food items and associated rankings. After the RAFIL adjustment is completed, control goes back to step S1603 where the system waits for User 140's next interaction.

Now, if the answer to the question in step S1604 was affirmative; meaning that User 140 would like to perform some physical activities, control will go to step S1605, where the Advisor system provides exercise or physical activity recommendations based on User 140's capabilities and preferences and computes the energy that would be burnt based on performing such exercises.

Alternatively, the Advisor system waits for User 140 to complete performing the physical activities and to enter the exact activity type and timing, which allows the Advisor system to provide a better estimate of the energy spent due to the performed exercises.

A third alternative is having User 140 wear a physical activity tracking Personal Health Device (PHD} 115, which automatically reports the energy spent to the Advisor User Platform 100.

Control then moves to step S1619 where User 140's personalized energy and nutrient budget CRDENIB will be adjusted to increase User 140's energy budget by an amount equal to the estimated amount of energy spent during the exercises. Additional foods or nutritional supplements containing minerals lost during exercise may also be recommended for consumption, causing the RAFIL to be changed.

An example of the Advisor systems' automatic change of the RAFIL as a result of physical activity, is shown in Table 10 (FIG. 17) where around 1 PM (right after lunch), User 140 walks; causing an estimated 150 K Calories to be burnt. It is shown in Table 10 that RAFIL Revision 2 is no longer valid and RAFIL Revision 3 is generated. This is the effect of adding 150 K Calories to User 140's CRDENIB energy budget, thus increasing User 140's budgets of carbohydrates, proteins, and fats which is translated to a potentially different combination of available foods, leading to RAFIL 3.

Another example of adjusting CRDENIB and RAFIL in response to reported physical activities is shown in Table 10 (FIG. 17) when User 140 visits the Fitness Center (FC) at 7 PM and reports burning 400 K Calories, causing step S1619 to increase CRDENIB's energy budget by 400 K Calories which would lead to the replacement of RAFIL revision 4 by RAFIL revision 5.

Step S1619 is also taken in response to User 140 and/or HCP 130's updates to CRDENIB, RAFIL, and possibly User 140's medication plan; as was discussed earlier in step S1508.

After CRDENIB and RAFIL are adjusted in step S1619, control goes back to step S1603 where the system awaits for User 140's next interaction.

If the answer to the question previously asked in step S1603, “Eating Now?” is affirmative, control moves to step S1608, which checks the Personal Usage History Store 114 to see if User 140 has taken any medications recently.

If the answer is yes, then control moves to step S1609, where the Advisor checks for any food items in the current RAFIL that may interact with the taken medications. If such food items exist in the current RAFIL, a blackout timer is set to temporarily remove the interacting food items from the current RAFIL until the interaction time window expires and an alert is sent to User 140 to withhold eating the interacting foods until further notice.

An example of this situation is shown in Table 8, drawing FIG. 13, where the metadata attribute TID for entry Grapefruit shows negative interactions between grapefruit and the medication Prograf if grapefruit is eaten less than two hours before or one hour after taking Prograf. To avoid this interaction, grapefruit is temporarily removed from the current RAFIL for one hour from the time Prograf was taken. After the 1-hour timer expires, grapefruit shows up as available in the RAFIL and User 140 receives an alert with an OK to eat grapefruit if desired. After all medications are accounted for by setting the appropriate timers, control advances to step S1610.

In step S1610, the advisor system inquires if User 140 is sick at the present time. If the answer is No, control advances to step S1612. However, if the answer is affirmative and User 140 is indeed sick, control moves to step S1611.

Alternatively, User 140 may inform the Advisor system at anytime that he is sick, utilizing the User Interface 210 block 217 of Advisor User Platform 100 and control is then transferred to step S1611.

In step S1611, the system asks User 140 for the type of sickness he has. This can be the common cold, influenza, or any other acute ailment requiring a special diet. The RAFIL items are adjusted to have food items appropriate for User 140's sickness taking into account any chronic diseases. The RAFIL content adjustments can be made automatically by consulting a Medical and Nutrition Knowledge Database (MNKDB) 107, or by consulting with an HCP 130 who can remotely make the necessary diet changes, or are done manually by User 140 if he has adequate self-management training. These options are programmable variables that are personalized by HCP 130 for each User 140 during the provisioning of the user's personal Advisor User Platform 100.

Control is then transferred to step S1612, where the Advisor system presents User 140 with food items, recipes, and remaining CRDENIB budgets, as shown in the following sections.

1. RAFIL Items available for consumption are displayed based on current location & remaining energy and nutrient balances.

User 140's current location is obtained through communicating with Advisor User Platform 100, which gets the location information by communicating with GPS servers 123 over GPS network link 124. Once User 140's location is determined, the Advisor determines which food items are available at the present location for User 140's consumption.

Table 9 (drawing FIG. 14) shows a sample RAFIL with food items available at User 140's home. The High-ranking items, such as Milk and all fruits and vegetables are presented to User 140 in bold italic font, while medium ranked items (such as Cheese Burger, Ranch Dressing, and Mac & Cheese) are displayed in regular font, and low ranking items as well as unavailable items (such as Ice Cream, Butter, and Pizza) are grayed out.

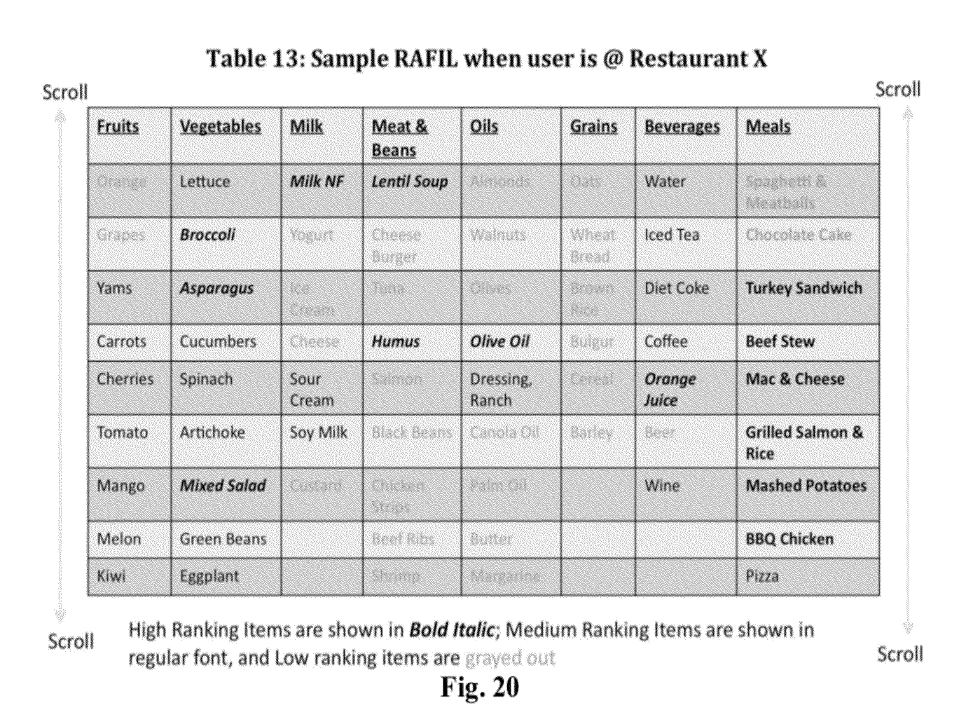

If the Advisor detects that User 140 is at Restaurant X, the Advisor will access Restaurant X's current menu and extract those menu items that provide the best nutrition for User 140 while not exceeding any of the CRDENIB components. Table 13 (FIG. 20) shows a sample of the RAFIL items when User 140 is in Restaurant X.

Alternatively, User 140 can request that the Advisor system recommend one or more of the neighboring restaurants (close to User 140's current or target location) with the most appropriate menu items to fit User 140's remaining CRDENIB budget. In this case, the Advisor will search all surrounding restaurants current menus, analyze the nutrient contents of their various menu items and recommend the top few items with the closest match to User 140's nutritional needs and remaining CRDENIB budgets. To estimate the nutrient contents of each restaurant menu items, the Advisor system checks information published by the restaurants and one or more of Food Nutrient Content and Physical Activity Databases 108.

2. Display remaining Nutrient Budget balances to enable User 140 to proactively participate in planning his next meal. If needed, User 140 can go shopping to increase the variety of Available food items that contribute to reaching the budgets for the required CRDENIB components. This information is also beneficial in motivating User 140 to exercise some more in order to gain energy credit resulting in increased food item selections.

3. Recommend recipes with food Items highest in remaining nutrients. This will assist User 140 in meeting his nutrient budgets. Recipes targeted for specific chronic disease patients' needs are made available in Educational & Motivational Multimedia Content Library 109.

Alternatively, a custom recipe creation application can be developed and deployed by the Advisor. The recipe creation application would take User 140's health condition, remaining CRDENIB and current RAFIL as inputs and would create one or more recipes containing the highest ranked food items with a balanced mix of the most needed nutrients.

4. Adjust RAFIL item portions to not exceed CRDENIB limits. This step is particularly necessary for weight management as it maintains the balance between meeting the recommended intake of nutrients without exceeding User 140's maximum energy budget.

Once the appropriate RAFIL items and recipes are selected for presentation to User 140, control advances to step S1613 where User 140 makes his selections of which food items and portions he would like to consume.

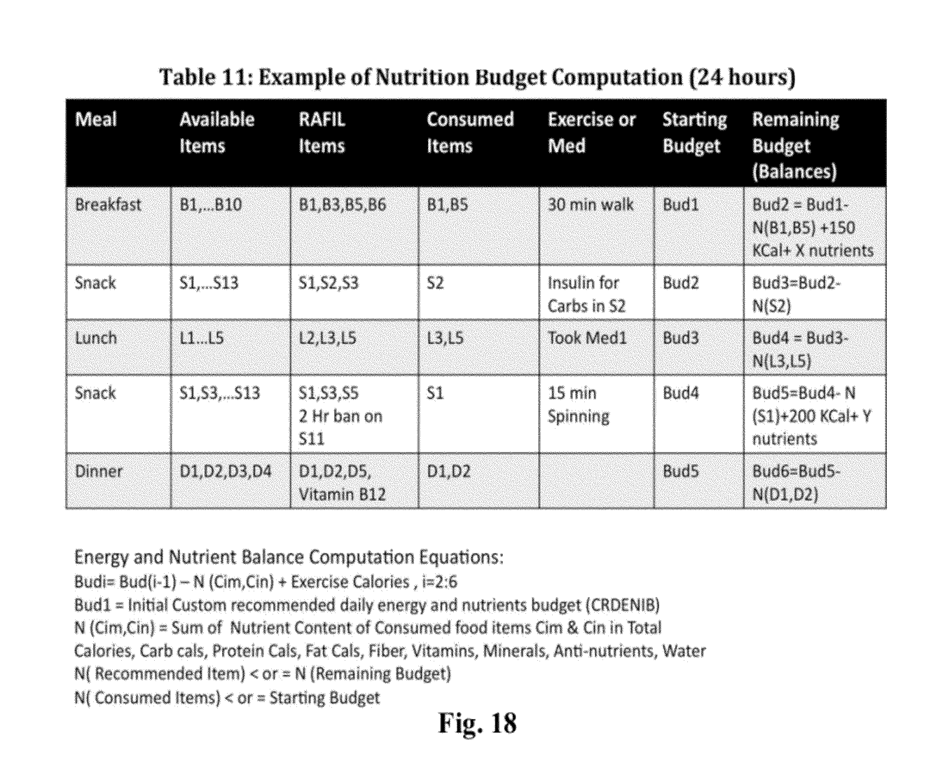

In step S1614, the Advisor system computes the total energy and nutrient values contained in the selected food items by utilizing information in Food Nutrient Content & Exercise Database 108. The remaining CRDENIB component budgets are computed by subtracting the computed energy and nutrient values of the food items to be consumed from the current CRDENIB values, as shown in Table 11 (drawing FIG. 18).

Now, in step S1615, the Adviser system checks if consuming the food items selected by User 140 in step S1613 would cause any of the components of CRDENIB to be exceeded. If the answer is affirmative, control will advance to step S1618 where User 140 will be prompted to adjust his selection and/or portion, which he does in step S1613.

Alternatively and preferably, visible (or audible) feedback on the User Interface 210 of Advisor User Platform 100 can be given to User 140 during the selection of each food item and portion he plans to consume (entered in process step S1613) showing the remaining nutrient budget balances and alerting him if any CRDENIB components are exceeded. He then cancels one or more selections, or adjusts the selected food item portions, until the Advisor system shows that these selections are acceptable. The Advisor system can also assist User 140 in making the appropriate selections by recommending combinations of food items that would not exceed the prescribed energy and nutrient budgets, and by recommending the previously mentioned pre-computed recipes.

If however, the Advisor system determines in step S1615 that consuming the items selected by User 140 in step S1613 will not cause any CRDENIB component values to be exceeded, control advances to step S1616 where the system approves User 140's selections. It also displays the total nutrient contents of the food items approved for consumption by User 140. This information is very useful for patients who must take certain dosages of medications based on the contents of the consumed food items. An example is insulin-dependent diabetics who need to know the amount of carbohydrates contained in each meal or snack in order to calculate the appropriate dosage of insulin they should take.

Finally, the Advisor system stores a copy of the food item metadata (e.g. photos, recipes, medication interactions, ranks, etc. . . . ) and nutrient composition in User 140's Advisor User Platform 100's Personal Usage History Store 104. This information will be useful for HCP 130's tracking of User 140's eating habits and having a local copy of the consumed food items' nutrients and metadata will speed up the CRDENIB and RAFIL computations when User 140 decides to consume the same food items in the future.

The remaining CRDENIB and RAFIL are adjusted in step S1617 to account for the consumed food items. An example of an embodiment of the CRDENIB computation steps and RAFIL item adjustments are shown in Table 11 (drawing FIG. 18). Referring to the Breakfast row of Table 11, we see that food items B1 through B10 are available for consumption. Based on the Advisor system analysis and ranking of all items, the highest-ranking items, B1, B3, B5, and B6 are selected for presenting to User 140 in the RAFIL. The next column shows that User 140 consumed items B1 and B5 followed by a 30-minute walk. The Advisor then adjusts the New Day CRDENIB by subtracting from it the nutrient values of B1 and B5 it looks up in Table 7 (FIG. 12).

Additionally, the Advisor keeps track of the physical activities performed by User 140 and estimates the energy burnt by the 30-minute walk at 150 K calories and adds it to the remaining CRDENIB budget, as shown in Table 8's Remaining Budget column.

Control advances to step S1620 on FIG. 16B, where the Advisor checks if there are remaining calories (energy) in today's CRDENIB. If the answer is no, meaning that User 140 has consumed all budgeted calories for today, the next check is made in step S1624 where the system checks if User 140 has consumed the minimum amounts of micronutrients (e.g. vitamins and minerals). If the answer is affirmative, then User 140 has met his daily requirements of energy and nutrients. Control advances to step S1629 where User 140 is informed that he has met his daily goals for today.

Finally, in step S1630 the Advisor system prepares a New Day RAFIL for use on the next day and execution halts at step S1631.

If, however, User 140 has not consumed all the required micronutrients for the day, the answer to the question in step S1624 would be negative and control advances to step S1625.

In step S1625, the Advisor system knows that User 140 needs to consume more micronutrients in order to meet the preset individualized nutritional goals, but the problem is that he has already consumed all the allocated calories in CRDENIB, as was discovered in step S1620. Since any food items he would need to consume in order to fulfill the micronutrient requirements are bound to contain some calories, the system asks User 140 if he is willing to perform some physical activities in order to earn energy credit for the food items containing the required micronutrients by spending some of his stored energy through exercise.

If User 140 agrees to exercise and earn the calories needed, control goes back to step S1605 on FIG. 16A, and after User 140 performs the exercise, CRDENIB is adjusted with the additional calories and User 140 is given another chance to meet his micronutrient requirements by going through the whole food selection process starting with step S1603.

If, however, User 140 is not willing or unable to perform any exercises at the present time, control moves to step S1626 where the system scans the RAFIL and attempts to find the food items with the lowest amount of calories, which would meet User 140's micronutrient daily budget; such as nutrient-rich vegetables and fruits.

Control is then transferred to step S1627 where the Advisor checks if the search performed in step S1626 for the low-calorie and micronutrient-rich food items was successful. If it was successful, control advances to FIG. 16A step S1603, where User 140 will have more chances to consume additional food. If no food items were found to provide the remaining micronutrient components, control advances to step S1628.

In step S1628, User 140 gets the recommendation to take a dosage of micronutrient supplements close to the CRDENIB amount or amounts of micronutrients he was not able to obtain through consuming natural foods.

Control then advances to step S1629, where User 140 is informed that he has met his daily goals for today.

Finally, in step S1630 the Advisor system prepares a New Day RAFIL for the next day and execution halts at step S1631.

Going back to step S1620, if User 140 has not consumed all allocated calories, the system checks in S1621 if any nutrients (e.g. protein, carbohydrates, fats, vitamins, or minerals) have reached their upper allowed limits (as computed in step S404). If the answer is No, control advances to step S1623.

If, however the answer is affirmative, the system advances to step S1622, where the system removes from the RAFIL any food items that contain those nutrients whose upper limits have been reached.

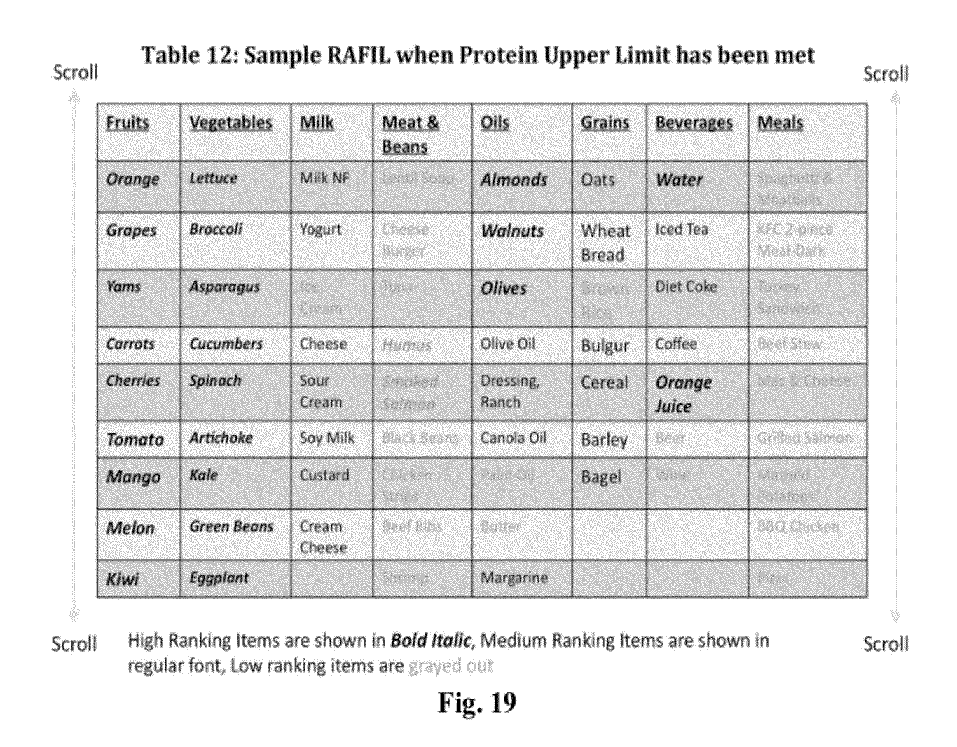

An example is given in Table 12 (drawing FIG. 19), which shows an adjusted RAFIL after User 140 has consumed his allocation of the nutrient protein. We can see that the remaining highlighted items in Table 12 are fruits and vegetables, which do not contain significant amounts of protein.

An alarm is sent to User 140 to insure that he is aware that he should not consume any significant amounts of protein for the rest of the day. To assist User 140 further in complying with the Advisor's recommendation, recipes containing the remaining low-protein items are presented to User 140.

Control then advances to step S1623, where the RAFIL is adjusted to account for the remaining CRDENIB budgets. The following sections outline some examples of how RAFIL is adjusted after User 140 consumes each meal or snack.

Remove or reduce portions of any items in RAFIL which if consumed as currently presented in RAFIL, would cause one or more CRDENIB components' (e.g. Calorie budget and/or any nutrients Upper Limits) upper limits to be exceeded. For example, if User 140 has a daily upper limit budget of 158 grams of protein (per Table 4, Men on 1800 calorie budget), and the remaining protein budget is 20 grams, any food item containing more than 20 grams of protein will be removed from the RAFIL. Alternatively, the portions of items having more than 20 grams of protein can be adjusted to bring their protein contents to be less than 20 grams, making them acceptable items.

Remove items from RAFIL that have been fully consumed and adjust the RAFIL contents to compensate for the consumed items' nutrients. For example, if User 140 has just consumed the last serving of milk, the current RAFIL will be updated to remove milk as an available food item. Additionally, if milk was the only source of calcium amongst the High ranked items on the current RAFIL and the requirements for calcium for the day (as specified in CRDENIB) have not been met yet, the system will look for an alternative source for calcium in the lower-ranked available items and increase their ranking to High, in order to get User 140's attention when planning his next meal or snack. Alternatively, a Calcium supplement is recommended.

The remaining budgets of each RDENIB component are reviewed and if it seems that certain nutrient components' budget balances are still high, meaning User 140 did not consume sufficient amounts of the food items containing these nutrients, the system will find food items containing high amounts of the needed nutrients and increase their ranks in the current RAFIL. Foe example, if after the lunch meal is consumed, the system finds out that the remaining balances of magnesium and zinc are still high, it would increase the ranks of food items containing high amounts of magnesium (such as pumpkin seeds and seaweed) and zinc (such as oysters and many kinds of mineral-fortified cereals) and recommend recipes for preparing these foods. The goal is to attract user 140's attention and entice him to consume such foods in order to get closer to reaching his healthy nutritional goals.

After the current RAFIL items and ranks are adjusted in step S1623 control is transferred back to step S1604 in FIG. 16A and the whole food/exercise/medication management process starts again.

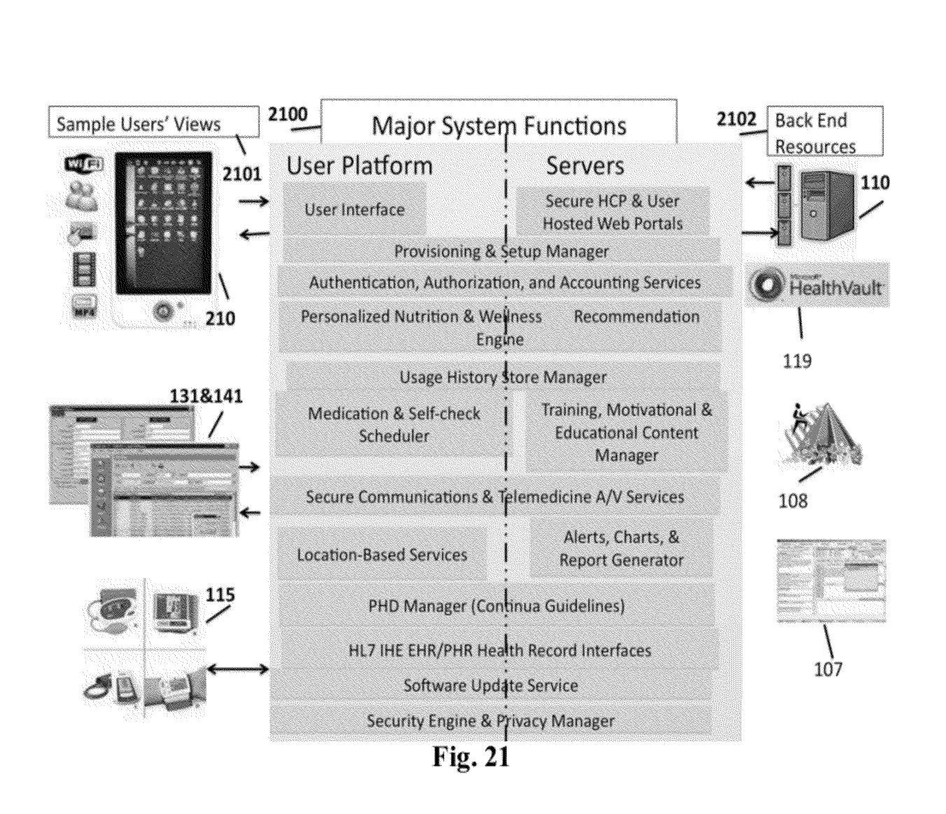

FIG. 21 shows an example of the major system components of one embodiment of the present invention.

The major system functions implemented by said embodiment of the present invention are distributed between the Advisor User Platform 100 and the Advisor Secure Servers 110 as shown in block 2100. The major functions shown perform the functions described in this specification and include Advisor User Platform User Interface, Advisor Secure Servers Secure HCP & User Hosted Web Portals, User Account and Platform Provisioning & Setup Manager, Authentication, Authorization, & Accounting (AAA) Services, Personalized Nutrition & Wellness Recommendation Engine, Usage History Store Manager, Medication and Self-check Scheduler, Training, Motivational, & Educational Content Manager, Secure Communications and Telemedicine A/V Services, Location-Based Services, Alerts, Charts, & Report Generator, PHD Manager, EHR and PHR Interfaces, Software Update Service, and Security Engine & Privacy Manager.

Sample Users' Views 2101 shows samples of interfaces and devices user 140 interacts with during his use of the Advisor system. Shown are examples of Advisor User Platform 100's Interactive User Interface 210, HCP and User Web Portals 131 and 141, and Personal Health Devices 115.

Back-end resources 2102 are comprised of Advisor Secure Servers 110, Personal Health Records 119, and various information databases utilized by the present invention; such as MNKDB 107 and Food Nutrient Content & Physical Activity Databases 108.

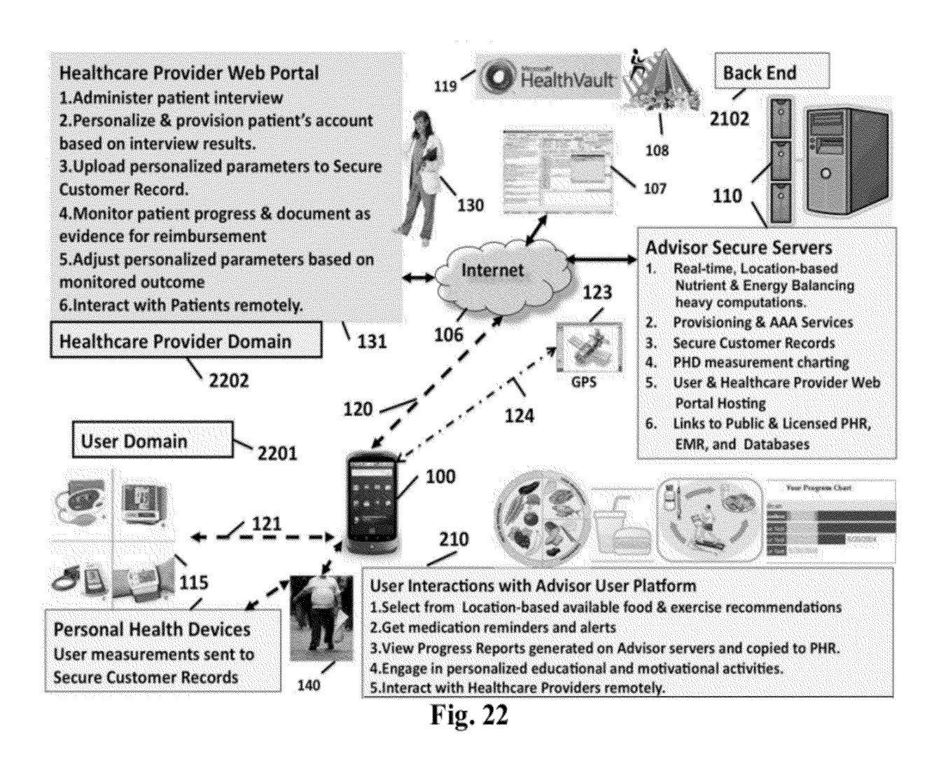

Finally, drawing FIG. 22 is a graphic representation of a typical user experience for a User 140 utilizing the benefits and features of a sample implementation of an embodiment of the present invention.

Looking at the Patient Domain 2201 we see User 140 interacting with his Advisor User Platform 100 (mobile device is shown here, but any other internet-connected device can be a host for the Advisor). Also shown in the Patient Domain 2201 is User 140's collection of Personal Health Devices (PHD) 115 which are connected to Advisor User Platform 100 over BAN/PAN/LAN network 121, which is comprised of wireless links such as Bluetooth, Zigbee, or Wi-Fi defined by several IEEE Standards.

Alternatively, PHD 115 can connect to User Platform 100 over a wired link such as Universal Serial Bus (USB).

As was previously explained in FIG. 15, when User 140 takes health-related measurements using PHD devices 115, the measurements are automatically sent to USER 140's Secure Customer Records 114 residing on Advisor Secure Servers 110 over the secure communications link 120.

To utilize the several health benefits of the present invention, which were detailed in the earlier sections of this disclosure, User 140 interacts with Advisor User Platform 100.

Examples of these interactions, which were described in detail in the previous sections, are shown in User Interface 210 and are listed here.

Select from Customized Location-based available food & Exercise recommendations.

Get medication reminders and alerts.

View Progress Reports generated on Advisor servers and copied to PHR. Engage in personalized educational and motivational activities. Interact w/Healthcare Providers remotely via secure audiovisual communications sessions.

In the Healthcare Provider Domain 2202, we see healthcare provider HCP 130 and a sample of activities she can perform remotely for managing User 140's health:

Administer patient interview to get User 140's personal information and health history. This can be preferably done on-line via electronic forms to facilitate updates and automated extraction of data.

Personalize & provision patient's account based on interview results and HCP 130's assessment of User 140's personal nutrition and wellness needs.

Upload personalized parameters to Secure Customer Records 114 for processing and storage by Advisor Secure Servers 110.

Monitor patient progress & document results as evidence for re-imbursement by insurance payers.

Remotely adjust User 140's personalized system parameters based on monitored data.

Interact w/Patients remotely via secure audiovisual communication sessions.

In the Back End 2102 resides Advisor Secure Servers 110, and multiple internet-connected databases such as PHR 119, EMR 118, MNKDB 107, and Food Nutrient Content & Physical Activity Databases 108.

The back end components provide the following computation and storage services to enable the realization of the present invention on low-cost, less capable Advisor User platforms 100:

Real-time, Location-based Nutrient & Energy Balancing heavy computations. Location is obtained through communicating with Advisor User Platform 100, which gets the location information through communicating with GPS servers 123 over GPS network link 124.

Provisioning & AAA Services to authorize User 140's Advisor User Platforms 100 secure access to the nutrition and wellness advice services.

Secure Customer Records storage and secure remote access services.

PHD measurement charting services for remote viewing by HCP 130 and User 140.

User & Healthcare Provider Web Portal Hosting services to enable remote viewing and updates to individual Secure Consumer Records.

Secure and fast links to Public & Licensed PHR, EMR, food, exercise, nutrient and medical knowledge databases to enable and expedite real-time decision making necessary for managing User 140's food, exercise, and medication timing and selection recommendations.

It is to be understood that the above described features can be achieved by a method in which a storage medium is supplied to a system or device, the storage medium having computer-executable instructions for realizing the above described operations, and a computing device (e.g., CPU and MPU) for the system or device that reads the computer-executable instructions stored in the storage medium and executes them.

In this case, the computer-executable instructions when read from the storage medium and performed by the computing device execute the operations of the above-described embodiments. Thus, the computer-executable instructions or the storage medium storing the computer-executable instructions therein constitute an embodiment.

As a storage medium for supplying the computer-executable instructions (e.g., a floppy disk, a hard disk, an optical disk, a magneto-optical disk, a CD-ROM, a CD-R, a magnetic tape, a non-volatile memory card, and a ROM) any applicable computer-readable storage medium can be employed.

When the computer-executable instructions are executed by a computing system, not only are the above-described operations of the embodiments realized, but also an operating system working on the computing system may carry out part or all of the actual processing that realizes the operations of the above-described embodiments.

The computer-executable instructions may be written to a memory provided on a function-extension board inserted into the computing device or on a function-extension unit connected to the computing device, and a CPU provided on the function-extension board or unit may carry out part of all of the actual processing that realizes the operations of the above described embodiments.

While the above disclosure describes illustrative embodiments, it is to be understood that the invention is not limited to the above disclosure. To the contrary, the invention covers various modifications and equivalent arrangements within the spirit and scope of the appended claims.

The embodiments of the Nutrition and Wellness Advisor and self-management method and system described herein are examples given to demonstrate the Advisor and specifically the Advisor recommendation engine's functionality and capability of providing comprehensive nutrition and wellness management services without relying on the user's memory, expertise, and removing the burden to keep records for measurements or food diaries.

Although an internet-based server was shown, the present invention can be practiced without the use of servers. Given sufficient execution power and storage capacity in the Advisor User Platform 100, the required databases and nutrient, exercise, and medication management decision making can be all embedded in the Advisor User Platform 100.

Conversely, the Advisor User Platform 100 can be a very thin client with very little computing and storage resources connected over very high-speed networks to remote servers 110, which provide most of the Advisor processing steps.

Although diabetes was used in many examples as the chronic disease managed by my Advisor method and system, the Adviser System can manage other chronic diseases, such as Congestive Heart Failure, obesity, cancer, and others. Even healthy individuals can benefit from the close activity tracking and personalized balancing of energy, nutrition, exercise, and if taken, medications provided by this innovative solution.

While the above disclosure describes illustrative embodiments, it is to be understood that the invention is not limited to the above disclosure. To the contrary, the invention covers various modifications and equivalent arrangements within the spirit and scope of the appended claims.

What I claim is:

1. A computer-assisted method for advising and assisting users in reaching and maintaining their health goals, the method comprising:
receiving, into the one or more computers, personal attributes and health information data for a specific user;
estimating, with the one or more computers, initial recommended daily energy and nutrients budgets for the specific user based on at least the user's personal attributes and health information data;
constructing and ranking, with the one or more computers, a personalized food item list populated with food items having cumulative energy and nutrient content to satisfy the present energy and nutritional needs for said user; wherein the food item list ranking is based at least on the user's recommended daily energy and nutrient budgets, the most recent user personal attributes, and health information data;
presenting, with the one or more computers, the ranked food item list to the user clearly distinguishing the different food item ranks to entice the user into selecting one or more food items having the highest ranks;
receiving, with the one or more computers, signals indicating the user's desire to consume one or more specific food items from the ranked food item list;
approving, with the one or more computers, the one or more user selections if their consumption will not result in exceeding the recommended daily energy budget or nutrient upper limits;
advising, with the one or more computers, the user to make food item selection adjustments if consuming the one or more specific food items would cause the energy budget or nutrient upper limits to be exceeded;
tracking, with the one or more computers, the user's actual food items and portions consumed and any performed health-related actions;
adjusting, with the one or more computers, the remaining daily energy and nutrient balances based on the user's consumed food items and any performed health-related actions; and
asynchronously updating, with the one or more computers, the contents of one or more of the recommended food item list, the food item ranks, and/or the recommended daily energy and nutrient budgets, in response to one or more of health-related user actions, external events, or physiological parameter measurements.

2. The method of claim 1, wherein the personal attributes include the user's age, weight, height, gender, ethnic background, genetic signatures, favorite and disliked foods, monetary food budget, favorite restaurants, and health goals.

3. The method of claim 1, wherein the health information data includes the user's medical health history; such as any chronic diseases, allergies, medications, and laboratory test results.

4. The method of claim 1, wherein the user health information data is stored in one or more electronic health records.

5. The method of claim 1, wherein the daily recommended micronutrient minimum requirements and upper limits are adjusted in reaction to the results of any physiological assessments of the current values of micronutrients present in the user's body; such that the minimum requirements are increased for any micronutrients the user's assessment shows as deficient and the upper limits are decreased for any micronutrients shown by the user's assessment to be excessive.

6. The method of claim 1, wherein the daily recommended micronutrient minimum requirements and upper limits are adjusted to take into account any known effects of one or more chronic diseases or one or more medications taken by the user on the retention or depletion of certain micronutrients in the user's body; such that the minimum requirements are increased for any micronutrients depleted due to the one or more medications or the one or more chronic diseases and the upper limits are decreased for any micronutrients retained due to the effects of the one or more medications or one or more chronic diseases.

7. The method of claim 1, wherein the recommended daily energy and nutrient budgets are continuously updated in reaction to at least the user's updated personal attributes and health information data.

8. The method of claim 1, wherein the food items list is populated with items which the user indicated are already available or can be made available for the user's consumption from financial and logistical points of view.

9. The method of claim 1, wherein the recommended food items list is constructed by using a Food Item Metadata Table.

10. The method of claim 1 wherein the physiological parameter measurements, the health-related activities, consumed macronutrients and micronutrients, and user's updated personal attributes and health information data are charted over periods of time for later viewing and analysis by the user, healthcare professionals, or automated data mining systems to help point out the causes for certain changes in health status indicators.

11. The method of claim 1, wherein one or more timers are set for predetermined periods of time to disallow certain food items from being presented to the user in the recommended food item list during the predetermined periods of time from taking certain medications, if said food items are known to have certain interactions with said medications; wherein the disallowed food items may become allowed upon expiration of said timers, as long as they do not later become disallowed due to energy budget or nutrient limitation constraints.

12. The method of claim 1, wherein alternative food items or adjusted portions of originally selected food items are recommended to the user if the consumption of one or more of the originally selected food items will result in exceeding the energy budget or one or more nutrient upper limits.

13. The method of claim 1, wherein nutritional supplements having values at least equal to any remaining balances in the minimum micronutrient requirements are recommended to the user when the daily energy consumption limits are reached before the minimum recommended amounts of micronutrients are consumed.

14. The method of claim 1, wherein the user is offered the options of performing certain physical activities or consuming certain amounts of micronutrient supplements in case the energy budget is consumed but the balance of micronutrients is still positive.

15. The method of claim 1, wherein the health-related actions are one or more of the:
food items consumed,
new food items acquired,
remaining energy and nutrient budget balances,
type, dose, and timing of medications taken,
user present location,
user intended destination,
physical activities performed,
user's specific acute sickness type,
user's physiological parameter measurements.

16. A computer-assisted method for estimating and updating recommended daily energy and nutrient budgets for a specific user comprised of:
receiving, into the one or more computers, ongoing updates of personal attributes and health information data for a specific user, wherein the health information data includes one or more personal health goals;
estimating, with the one or more computers, the recommended daily energy requirements for the specific user based on the received personal attributes and health information data;
estimating, with the one or more computers, the macronutrient energy distribution ratios based on the health information data and one or more personal health goals;
estimating, with the one or more computers, the recommended micronutrient requirements based on the health information data;
monitoring, with the one or more computers, the stored personal records for any changes in the user's personal attributes and health information data; and
updating, with the one or more computers, the estimated energy budget, ranges of macronutrient distribution ratios, and ranges of micronutrient requirements based on the values of the monitored changes.

17. The method of claim 16, wherein the health information data includes any chronic diseases the user has, or types, doses, and times of any medications taken by the user.

18. The method of claim 16, wherein the health information data includes physiological measurements of the user indicating numerical values of any deficiencies or excesses in certain micronutrients.

19. A system for assisting users in reaching and maintaining their nutrition and wellness goals, the system comprising:
one or more computer-readable storage devices; and
one or more processors configured to
receive ongoing updates of personal attributes and health information data for a specific user;
receive ongoing updates of recommended daily energy and nutrients budgets for the specific user;
construct and rank, with the assistance of the one or more computers, a personalized food item list populated with food items having cumulative energy and nutrient content to satisfy the present energy and nutritional needs for said user; wherein the food item list ranking is based at least on the user's recommended daily energy and nutrient budgets, the most recent user personal attributes, and health information data;
present the ranked food item list to the user clearly distinguishing the different food item ranks to entice the user into selecting one or more food items having the highest ranks;
receive signals indicating the user's desire to consume one or more specific food items from the ranked food item list;
approve the one or more user selections if their consumption will not result in exceeding the recommended daily energy budget or nutrient upper limits;
advise the user to make food item selection adjustments if consuming the one or more specific food items would cause the energy budget or nutrient upper limits to be exceeded;
track the user's actual food items and portions consumed and any performed health-related actions;
adjust the remaining daily energy and nutrient balances based on the user's consumed food items and any performed health-related actions; and
asynchronously update the contents of one or more of the recommended food item list, the food item ranks, and/or the recommended daily energy and nutrient budgets, in response to one or more of health-related user actions, external events, or physiological parameter measurements.

20. The system of claim 19, wherein the one or more processors are connected over one or more wired or wireless communications networks.

21. A computer-readable medium having computer executable instructions stored thereon,
the instructions being executable by one or more computing devices in order to cause the one or more computing devices to perform operations comprising:
receiving, into the one or more computers, personal attributes and health information data for a specific user;
estimating, with the one or more computers, initial recommended daily energy and nutrients budgets for the specific user based on at least the user's personal attributes and health information data;
constructing and ranking, with the one or more computers, a personalized food item list populated with food items having cumulative energy and nutrient content to satisfy the present energy and nutritional needs for said user; wherein the food item list ranking is based at least on the user's recommended daily energy and nutrient budgets, the most recent user personal attributes, and health information data;
presenting, with the one or more computers, the ranked food item list to the user clearly distinguishing the different food item ranks to entice the user into selecting one or more food items having the highest ranks;
receiving, with the one or more computers, signals indicating the user's desire to consume one or more specific food items from the ranked food item list;
approving, with the one or more computers, the one or more user selections if their consumption will not result in exceeding the recommended daily energy budget or nutrient upper limits;
advising, with the one or more computers, the user to make food item selection adjustments if consuming the one or more specific food items would cause the energy budget or nutrient upper limits to be exceeded;
tracking, with the one or more computers, the user's actual food items and portions consumed and any performed health-related actions;
adjusting, with the one or more computers, the remaining daily energy and nutrient balances based on the user's consumed food items and any performed health-related actions; and
asynchronously updating, with the one or more computers, the contents of one or more of the recommended food item list, the food item ranks, and/or the recommended daily energy and nutrient budgets, in response to one or more of health-related user actions, external events, or physiological parameter measurements.
